(19) United States
(12) Patent Application Publication
(10) Pub. No.: US 2019/0317115 A1
(43) Pub. Date: Oct. 17, 2019

(54) INTEGRATED POINT-OF-CARE CARTRIDGE ASSAY SYSTEM

(71) Applicant: Vitameter Inc., Kitchener (CA)

(72) Inventors: James Laughlin MacLean, Toronto (CA); Calvin James Brereton, Sudbury (CA); Nirushan Darren Udayakumar, Richmond Hill (CA); Lynnea Lurline Yee Lin Tan, Richmond Hill (CA); Nishi Pavankumar Bhatt, Kitchener (CA)

(73) Assignee: Vitameter Inc.

(21) Appl. No.: 16/381,721

(22) Filed: Apr. 11, 2019

Related U.S. Application Data
(60) Provisional application No. 62/655,949, filed on Apr. 11, 2018.

(51) Int. Cl. G01N 35/00; G01N 33/53; B01L 3/00

(52) U.S. Cl. CPC G01N 35/00029; G01N 33/5302; G01N 35/00722; B01L 3/5023; B01L 2200/16; G01N 2035/00108; G01N 2035/00891; B01L 2300/0825; B01L 2400/0406; B01L 3/52

(57) ABSTRACT

A method and system for user-friendly point of care assay is described. The cartridge utilizes a lateral flow assay, buffer chambers and capillary sample collection. The sample is first collected by the capillary tube attached to the cartridge. The cartridge is then actuated, drawing the sample into the lateral flow strip and introducing a buffer liquid into the system thereby automatically performing the assay. An electronic reader may be used to measure the results of the assay.

### CROSS-REFERENCE TO RELATED PATENT APPLICATION

This application claims the benefit of U.S. Provisional Patent Application No. 62/655,949, filed Apr. 11, 2018, and the entire content of U.S. Provisional Patent Application No. 62/655,949 is hereby incorporated by reference.

### FIELD

The present disclosure relates to a system and method for an easily operated system and method for quantification of an immunoassay. More specifically, the present disclosure encompasses systems and methods for more convenient sample collection, sample processing, simple conduction of an immunoassay in a unique orientation and quantification using an optical system designed for feasibility in a point-of-care setting.

### BACKGROUND

The diagnostic assay is a well-understood methodology that can be used to procedurally measure various target molecules or analytes, such as lipids, proteins, drugs (& other entities) in a variety of samples and matrices. An assay can include, but is not limited to, immunoassays, colorimetric, enzymatic, turbidimetry, and transmittance, for example.

This has particular importance and high use cases in the healthcare industry. An assay can be used to measure various biomarkers in a sample matrix, including blood, saliva, tears, sweat, urine, and feces. These measurements can be used to diagnose a plethora of conditions and diseases.

Assays come in various forms, but a conventional assay may require a doctor, a laboratory facility with technicians and other trained professionals. Assays typically take several hours to perform with multiple steps being performed by the technicians. Samples from patients are usually collected by trained professionals in a clinical setting, and the sample may require some post-collection processing such as blood clotting and filtering. Following this, the technician must perform the assay which may involve performing various steps to the processed sample, such as diluting with buffers or applying it to a separate apparatus.

Quantification is done with a large machine in a controlled laboratory environment. While this can be useful for high throughput screening, the amount of personnel, the amount of time involved in performing an assay, the laboratory space requirements, and the equipment required for quantification drives up the cost.

As such, point of care assays are becoming increasingly popular. Beyond traditional laboratory and clinical testing, great strides have been made towards point of care testing using assay technologies. These include, but are not limited to, lateral flow assays, lab-on-a-chip technologies, dipstick assays and colorimetric tests. An example of such a test is an at-home pregnancy test. However, the operation of the assay is very time-consuming with many steps, meaning that obtaining results can be quite slow, but more importantly that there is a lot of room for human error during the process. Each step adds the potential for this kind of error, making the results from these assays highly irreproducible when used by untrained people. As such, most point-of-care tests still require trained professionals to assist in sample collection due to its complexity in nature.

To distribute the technology of assays in a cost effective medium, lateral flow assays have been commercialized with numerous applications, such as consumer diagnostic devices. These assays are paper microfluidic devices that rely on capillary forces to induce fluid flow through porous mediums. The lateral flow assay strip is typically composed of four membranes: a filter membrane, a conjugate pad, a reaction membrane, and an absorbent pad. These membranes are adhered to a backing card for support and to maintain the orientation of the membranes with respect to each other. The detection region is present on the nitrocellulose, which encompasses the test line and the control line. The test line is used to provide the results of the test, while the control line insures that the test is functioning properly. For detection of the desired analyte, two formats are common for the lateral flow assay: the sandwich and competitive formats. Both of these formats can be performed in a one-step or two-step process.

One of the reasons the lateral flow assay is pervasive in point of care testing is the low cost of production. The flow system consists of a number of membranes that form different functions of the assay. The membranes are cut into long rectangles and carefully layered onto each other, with overlaps to allow fluid to flow sequentially from one membrane to the next. The batch is then cut into thin strips, perpendicular to the length of the membranes, such that it forms many identical strips from a single batch. In larger manufacturing processes, the membranes are spooled on reels and are continuously layered together and cut in an elaborate, but efficient, “reel to reel” system.

### SUMMARY

In one aspect, in accordance with the teachings herein, there is provided a test cartridge that allows for an automated assay of various analytes in a sample, wherein the test cartridge comprises a capillary assembly to receive, store and release the sample; a buffer capsule for storing and releasing a buffer liquid; a buffer membrane to receive the buffer liquid during the assay; a test strip that is coupled to the buffer membrane and adapted to perform the assay after receiving the sample and the buffer liquid; a test strip holder adapted to hold the test strip and buffer membrane and move towards the capillary assembly and the buffer membrane to provide the sample to the test strip and the buffer liquid to the buffer membrane when the assay is performed; and an enclosure for providing a housing for the test cartridge.

In at least some embodiments, the test strip holder is adapted to move from a pre-actuation position to a post actuation position, wherein in the pre-actuation position the test strip and the buffer membrane are spaced apart from the capillary assembly and the buffer capsule, respectively, and in the post-actuation position the test strip is brought into contact with the capillary assembly to receive the sample, the membrane strip is brought into contact with the buffer capsule to receive the buffer liquid which is then transferred to the test strip and the test strip performs the assay.

In at least some embodiments, the test cartridge further comprises a sliding rail system made of slots formed in one surface of the test strip holder and pegs formed in an opposing surface of the enclosure and the test strip holder is adapted to move along the sliding rail system.

In at least some embodiments, the enclosure comprises an aperture opposite a bottom end of the test strip holder for receiving a force to move the test strip holder from the pre-actuation position to the post-actuation position.

In at least some embodiments, the capillary assembly is located at an upper corner of the enclosure and comprises an outer facing end having a first aperture open to an exterior of the enclosure; an inner facing end having a second aperture open to an interior of the enclosure; and a capillary tube between the outer and inner facing ends, the capillary tube drawing the sample into the capillary assembly using capillary forces during use.

In at least some embodiments, the test strip comprises: a backing card for providing support for membranes of the test strip; a reaction membrane disposed on the backing card, the reaction membrane having reaction zones including a control zone for providing control measurements and at least one assay reaction zone for reacting with at least one analyte of interest; a conjugate pad overlaid on a distal portion of the reaction membrane, the conjugate pad containing chemicals for performing the assay; and a sample pad overlaid on a distal portion of the conjugate pad and wrapped around a back surface of the backing card and having a rounded end at a distal portion of the test strip, the sample pad having a filter membrane disposed adjacent the rounded end for receiving the sample and having a buffer entrance area disposed near the back surface of the backing card for receiving the buffer liquid.

In at least some embodiments, the chemicals of the conjugate pad comprise at least one of buffers, surfactants, salts, antibodies and labelling agents.

Generally in the embodiments, when the test strip holder is moved to the post-actuation position, the rounded end of the sample pad is moved to the inner facing end of the capillary assembly to receive the sample from the capillary tube.

In at least some embodiments, the buffer membrane comprises a buffer intake area to receive the buffer liquid from the buffer capsule; a U-shaped bottom region downstream of the buffer intake area where the buffer liquid pools as the buffer liquid drawn from the buffer capsule; a junction region downstream of the bottom region and adjacent to the test strip; and an exit region comprising an arm adjacent to and downstream of the junction region, the exit region overlapping with the buffer entrance area of the test strip to provide the buffer liquid thereto.

In at least some embodiments, the test strip holder comprises a protrusion that is moved into an end of the buffer capsule to release the buffer liquid to the buffer membrane when the test strip holder is moved to the post-actuation position.

In at least some embodiments, the buffer membrane comprises an arm portion that is fluidly coupled to the test strip to provide the buffer liquid thereto, the arm portion having a size, shape and material that are selected to provide a desired flow rate to delay the arrival of the buffer liquid into the test strip.

In at least some embodiments, the enclosure comprises an optical window that is aligned with the reaction zones of the reaction membrane when the test strip holder is moved to the post-actuation position to allow for optical measurements of the reaction zones during assay results measurement.

In at least some embodiments, the enclosure comprises a first indicator to indicate when the test cartridge is new and when it is used.

In at least some embodiments, the enclosure comprises a second indicator to indicate when the test cartridge is expired.

In at least some embodiments, the enclosure comprises a third indicator to indicate when the test cartridge is exposed to prohibited humidity or temperature during storage.

In at least some embodiments, the enclosure comprises first and second enclosure pieces to provide a housing for the test cartridge to protect the test strip from contamination or exposure to environmental humidity.

In at least some embodiments, the test cartridge further comprises an NFC tag disposed with the enclosure, the NFC tag being adapted to store information about the test cartridge including information about at least one of a type of assay that is performed by the test cartridge and parameters that are used to determine the assay results from assay measurements.

In at least some embodiments, the test strip and buffer membrane are disposed within the test cartridge to perform a lateral flow assay.

In another aspect, in accordance with the teachings herein, there is provided an electronic reader for performing an assay test and measuring test results, wherein the electronic reader comprises: a slot for receiving a test cartridge that is defined according to any one of claims 1 to 18; an optical system for obtaining optical measurements from the test cartridge; and a microprocessor that is coupled to the optical system, the microprocessor being adapted to control the electronic reader, receive the optical measurements and determine assay results from the optical measurements.

In at least some embodiments, the cartridge further comprises a tab in the slot that provides a force to move the test strip holder of the test cartridge to begin the assay when the test cartridge is inserted into the slot thereby allowing for single user action for performing the assay and measuring the assay results.

In at least some embodiments, the slot is vertical to provide a vertical orientation for the test cartridge which aids in movement of the sample along the test strip and the buffer liquid along the buffer membrane and the test strip.

In at least some embodiments, the electronic device further comprises a display to show the assay results and/or error information when an error is encountered during the assay or operation of the electronic reader.

In at least some embodiments, the electronic device further comprises an NFC reader for reading information about the test cartridge including at least one of a type of assay that is performed by the test cartridge and parameters that are used to determine the assay results from assay measurements.

In at least some embodiments, the electronic device further comprises a communication module for pairing and/or communicating with an application of an external device for sending the assay results and associated assay test information.

In at least some embodiments, the communication module is adapted to use a Bluetooth or WiFi communication protocol.

In another aspect, in accordance with the teachings herein, there is provided a kit for performing an assay for point of care testing wherein the kit comprises at least one test cartridge that is defined according to any one of the test cartridge embodiments described herein and an electronic reader that is defined according to any one of the electronic reader embodiments described herein.

In another aspect, in accordance with the teachings herein, there is provided a method of measuring a sample with at least one analyte of interest, wherein the method comprises: providing a sample to a test cartridge having a test strip and a buffer membrane for performing an assay on the sample to detect the at least one analyte of interest; inserting the test cartridge into an electronic reader which provides an actuation force to initiate the assay in the test cartridge; measuring assay results using the electronic reader; and displaying, storing and/or sending the assay results using the electronic reader.

The test cartridge that is used for performing the method may be defined according to any one of the test cartridge embodiments described herein.

The electronic reader that is used for performing the method may be defined according to any one of the electronic reader embodiments described herein.

The method of performing the measurement may have various alternatives and steps as described in the various method embodiments described herein.

Other features and advantages of the present disclosure will become apparent from the following detailed description taken together with the accompanying drawings. It should be understood, however, that the detailed description and the specific examples, while indicating preferred embodiments of the teachings herein, are given by way of illustration only, since various changes and modifications within the spirit and scope of the teachings herein will become apparent to those skilled in the art from this detailed description.

### BRIEF DESCRIPTION OF THE DRAWINGS

For a better understanding of the various example embodiments described herein, and to show more clearly how these various example embodiments may be carried into effect, reference will be made, by way of example, to the accompanying drawings which show at least one example embodiment, and which are now described. The drawings are not intended to limit the scope of the teachings described herein.

FIGS. 1A-1B shows an example embodiment of a lateral flow test strip in a folded and unfolded configuration, respectively, in accordance with the teachings herein, where the test strip can be integrated into a point of care device.

FIG. 1C shows a top view of the lateral flow test strip in the unfolded configuration shown in FIG. 1B.

FIG. 1D shows a side view of the lateral flow test strip in the folded configuration shown in FIG. 1A.

FIGS. 2A-2B show an isometric view of an example embodiment of an assembled cartridge system and an exploded view of that system, respectively, in accordance with the teachings herein, that can be integrated into a point of care device.

FIG. 3A is a bottom view of the cartridge of FIG. 2A.

FIGS. 3B-3C are cross-sectional views showing the cartridge of FIG. 2A in a pre-actuation state before actuation and in a post actuation state, respectively, in accordance with the teachings herein with part of the enclosure and test strip holder removed.

FIGS. 4A-4B show isometric views of the cartridge of FIG. 2A in the pre-actuation and post-actuation states, respectively, with part of the enclosure and test strip holder removed.

FIG. 5 shows an isolated view of the components involved in liquid flow include the test strip, buffer membrane, capillary and buffer capsule in accordance with the teachings herein.

FIGS. 6A-6B show isometric and front views of a first test strip holder part in accordance with the teachings herein.

FIGS. 6C-6D show isometric and front views of a second test strip holder part in accordance with the teachings herein

FIGS. 7A-7C show isometric, front, and side views of an example embodiment of the capillary tube, in accordance with the teachings herein, that can be integrated into a point of care test cartridge.

FIGS. 8A-8B show front and side views of the cartridge of FIG. 2A.

FIGS. 8C-8D show a longitudinal cross-sectional view of the cartridge along the test strip and a transverse cross-sectional view of the cartridge cut through a reaction zone, respectively.

FIGS. 8E-8F are enlarged views of portions of the cross-sectional views of FIGS. 8C-8D, respectively, to show how light interacts at the optical opening of the cartridge.

FIGS. 9A-9B show an example embodiment of the test indicator in the pre-actuation state of the cartridge and the test indicator in the post-actuation state of the cartridge, respectively, indicating its use, in accordance with the teachings herein.

FIG. 9C shows the cartridge of FIG. 9B in the post-actuation state with a piece of the cartridge enclosure removed.

FIG. 10 is a block diagram of an example embodiment of a test system comprising an electronic reader and a cartridge where the electronic reader can actuate the test cartridge to begin the assay and measure the test results for a cartridge in accordance with the teachings herein.

FIGS. 11A-11B show an example embodiment of a device-cartridge system prior to cartridge insertion and after the cartridge has been inserted, respectively, in accordance with the teachings herein.

FIGS. 12A-12B show cross sectional views of the device-cartridge system shown in FIGS. 11A-11B, respectively.

FIGS. 13A-13B show the optical sensor assembly with the PCB, optics and electrical components in an isometric and cross-sectional view, respectively, in accordance with the teachings herein.

FIG. 14 is a flow chart of an example embodiment of a method of performing an assay using a cartridge and an electronic reader in accordance with the teachings herein.

FIG. 15 shows the collection of a blood sample with the capillary of the cartridge system in accordance with the teachings herein.

FIGS. 16A-16G show perspective, front, back, left side, right side, top and bottom views of an example embodiment of a cartridge in accordance with the teachings herein.

FIGS. 17A-17G show perspective, front, back, left side, right side, top and bottom views of an example embodiment of an electronic reader in accordance with the teachings herein.

FIGS. 18A-18G show perspective, front, back, left side, right side, top and bottom views of an example embodiment of the cartridge of FIGS. 16A-16G being used with the electronic reader of FIGS. 17A-17G in accordance with the teachings herein.

FIGS. 19A-19C show front, bottom, and back views of an example embodiment of a cartridge in accordance with the teachings herein.

FIG. 19D shows a cross sectional view of the cartridge of FIGS. 19A-19C taken along line C-C of FIG. 19B.

FIG. 20A shows a top view of an embodiment of an electronic reader in accordance with the teachings herein.

FIG. 20B shows a cross sectional view of the electronic reader of FIG. 20A taken along line B-B of FIG. 20A.

FIG. 20C shows a cross sectional view of the electronic reader of FIG. 20A with the cartridge of FIGS. 19A-19D inserted therein.

The skilled person in the art will understand that the drawings, further described below, are for illustration purposes only. The drawings are not intended to limit the scope of the applicant's teachings in any way. Also, it will be appreciated that for simplicity and clarity of illustration, elements shown in the figures have not necessarily been drawn to scale. For example, the dimensions of some of the elements may be exaggerated relative to other elements for clarity. Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to assays or methods having all of the features of any one of the assays or methods described below or to features common to multiple or all of the assays or methods described herein. It is possible that there may be an assay or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or fluid connotation and indicate that two elements can be directly connected to one another or connected to one another through one or more intermediate structural elements or fluid flows, depending on the particular context.

It should also be noted that, as used herein, the wording “and/or” is intended to represent an inclusive-or. That is, the text “X and/or Y” is intended to mean X or Y or both, for example. As a further example, the text “X, Y, and/or Z” is intended to mean X or Y or Z or any combination thereof.

It should be noted that terms of degree such as “substantially”, “similarly”, “about” and “approximately” as used herein mean a reasonable amount of deviation of the modified term such that the end result is not significantly changed. These terms of degree may also be construed as including a deviation of the modified term, such as 1%, 2%, 5%, or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as up to 1%, 2%, 5% or 10%, for example.

A lateral flow test kit can contain several separate components including a sample collector, a sample preparation buffer, a buffer capsule, and a test strip. It may take from 1 to 20 minutes to complete an assay with multiple steps and wait times with conventional test kits. For the diagnostic industry, the ability to use a test kit so that the test is performed correctly without error restricts many test kits for clinical use as they are too complicated for an untrained user. However, even in a clinic, a practitioner does not want to spend valuable time and effort operating a test kit.

An ideal test kit would require a low amount of user interaction and simple operation. Advantageously, in accordance with the teachings herein, the separate kit components mentioned above are brought together and integrated into a single physical unit, removing the need for a multistep kit. This is done by enclosing these separate components in a functional cartridge such that an assay is performed within the cartridge after a sample is provided to the cartridge and the cartridge is actuated. The test can be operated in less than a minute with results collected automatically by an accompanying device, i.e. an electronic reader, within 10 minutes.

In a first aspect, in accordance with the teachings herein, at least one embodiment of a test cartridge, an analyzer device (i.e. electronic reader) and a membrane-based test strip is provided herein.

In at least one embodiment, a user inserts a sample into the cartridge by holding a drop of the sample next to a sample collection stage that includes a capillary assembly with a capillary tube entrance for drawing the sample into the test cartridge.

In at least one embodiment, the test cartridge is then inserted into the electronic reader to begin the assay. The act of pushing the test cartridge into the electronic reader actuates a test strip holder, which results in the sample being pulled from the capillary tube into the membrane-based test strip and releases the liquid reagents (i.e. buffer liquids) to the membrane-based test strip to begin the assay.

In at least one embodiment, the electronic reader measures the result of the assay after the reaction has been completed and displays the result on a display associated with the electronic reader.

In at least one embodiment, the test cartridge is disposable.

In at least one embodiment, the membrane-based test strip comprises layered membranes that are laminated with respect to one another. The membranes are dimensioned and treated with chemicals so that together they facilitate an assay when the liquid sample is applied to the test strip. The test strip can include chemicals for chemically treating, filtering and/or reacting with the sample. A common example of this is a lateral flow immunoassay. In this embodiment, the test strip is bent during the manufacturing process to create fluid flow on both sides of the test strip. This allows the sample liquid to be applied directly to the end of the test strip. It also allows the assay to be performed at different orientations without interfering with sample intake. This test strip may also be held within a cartridge system for stability and added functionality.

In at least one embodiment, the test cartridge consists of an enclosure, a capillary tube, a test strip holder, the assay test strip, the buffer channel membrane, a buffer capsule, an NFC tag and a test indicator.

In at least one embodiment, the enclosure of the test cartridge protects the test strip from contamination from the environment or the user's hands. The enclosure also helps protect the test cartridge against environmental humidity, which can hinder the test strip's performance because the humidity makes the flow less predictable within a timespan of minutes and allows the antibodies to degrade in hours if the test strip is left completely open to the environment.

In at least one embodiment, the NFC tag and the test indicator identifies the cartridge to the user and the device. It also encodes information used to interpret the chemical reaction that is passed onto the device. For example, the encoded information may include parameters for building a reference curve and indicating what type of test is performed by the test cartridge. The test indicator may be used to indicate when the test cartridge has been used. For example, the test indicator can be a sticker which may have different colored areas. The structural arrangement of the test cartridge may also be used to indicate if it is a used or a new test cartridge. For example, a new cartridge that just received a sample may make a clicking sound when it is inserted into the electronic reader (described below) for measuring the test results.

In at least one embodiment, the test strip holder has multiple functional areas that are described herein, including a buffer fang, a buffer capsule, buffer channel membrane positioning elements, test strip positioning elements, a detent tab and optical windows. The buffer capsule holds the buffer liquid that is wicked up by the test strip and pushes the sample through the test strip. The buffer liquid may contain buffering agent, salts and other chemicals used for the assay. The buffer fang is a pointed arm on the test strip holder that punctures the buffer capsule that is in the test cartridge to release the buffer liquid to the buffer channel membrane. The buffer channel positioning elements on the test strip holder encloses a buffer membrane through which the buffer passes. The detent tab and arm help hold the test strip holder in place and control the movement of the test strip holder within the test cartridge. In particular, the test strip holder positions the test strip for operation and precise measurement. The optical windows may be cutouts in the test strip holder to allow for optical measurement of the reaction on the test strip.

In at least one embodiment, the capillary tube draws in a sample, such as blood from a user's finger, and controls the volume of the sample that is transferred to the test strip. A capillary tube has been used in other POC diagnostic assays. However, the use of the capillary tube has been a poor user experience in conventional assays. For example, in conventional devices the capillary tubes are tedious to hold as they are small, breakable and prone to accidental release of the fluid. In addition, in conventional assays it is possible to only partially fill up the tube or only partially empty it, resulting in improper usage and erroneous test results. The use of these conventional assays also feels like using a lab kit and complicates the steps needed to perform the assay, which opens up the conventional system to user error and infrequent use. In contrast, in accordance with the teachings herein, a sample collection stage and test system is cleanly integrated into a single test cartridge with various features for improving the user experience. For example, integrating the capillary tube into the test cartridge in a user-friendly manner, in accordance with the teachings herein, avoids the user from having to hold another piece of test equipment as they conduct the test. The user can simply lance their finger and bring the capillary tube to their finger to provide the sample to the test cartridge. The test cartridge can then be inserted into the electronic reader, which then automatically begins the test and measures the test results.

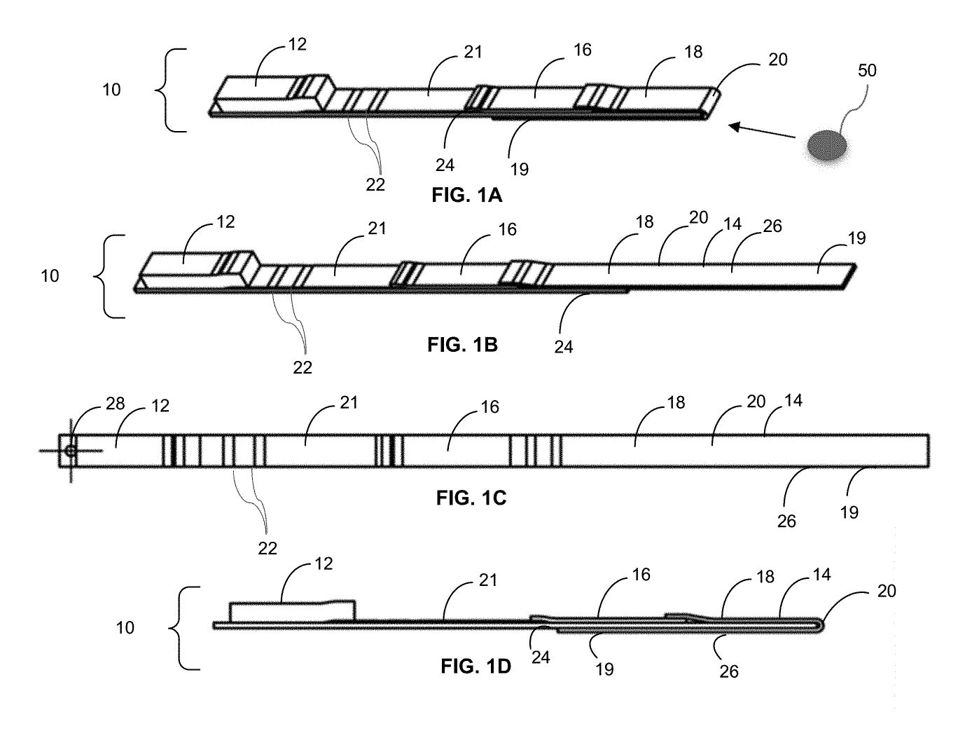

FIGS. 1A-1D show an example embodiment of a test strip 10 that is a lateral flow strip that can be used to perform an assay. The format of the test strip 10 and its dimensions vary based on the particular assay that it will perform. The test strip 10 comprises several membranes that are arranged in a particular configuration with certain overlapping regions. For example, the test strip 10 comprises an absorbent pad 12, a sample pad 14, a conjugate pad 16, a filter membrane 18, a buffer entrance area 19, an end portion 20, a reaction membrane 21, reaction zones 22, a backing card 24, and a back surface 26. The backing card 24 provides a substrate for the test strip 10. For descriptive purposes, the right most end of the test strip 10 is called the distal end or distal direction and the left most end of the test strip 10 is called the proximal end or proximal direction.

The reaction membrane 21 is placed on a proximal portion of the backing card 24 spaced apart by a first distance from the proximal end of the backing card 24. The distal end of the absorbent pad 12 overlaps a proximal end portion of the reaction membrane 21. The reaction membrane 21 is disposed downstream of the conjugate pad 16. The reaction membrane 21 includes reaction zones 22, which can be used to measure the results of the assay, as is described in further detail below.

The absorbent pad 12 is disposed near the proximal end of the backing card 24 such that the absorbent pad 12 is spaced apart by a second distance from the proximal end of the backing card where the second distance is smaller than the first distance. The absorbent pad 12 is disposed downstream of the reaction membrane 21. An aperture 28 is near the proximal end of the backing card 24 downstream of the absorbent pad 12.

The conjugate pad 16 is disposed on a medial portion of the backing card 24. A proximal end portion of the conjugate pad 16 overlaps a distal end portion of the reaction membrane 21 and the distal end of the conjugate pad 16 is spaced apart from the distal end of the backing card 24 by a third distance. The conjugate pad 16 is disposed downstream of the filter membrane 18.

The sample pad 14 is disposed at the distal end portion of the backing card 24. The distal end of the sample pad 14 extends past the distal end of the backing card 24 (see FIG. 1B), which allows the sample pad 14 to be folded so that the sample pad 14 covers both the front and rear surfaces of the distal end portion of the backing card 24. The portion of the sample pad 14 that folds over to cover the rear surface of the backing card 24 provides a rear surface for the test strip 10. The portion of the sample pad 14 that is opposite the conjugate pad 16 is the buffer entrance area 19, which receives buffer liquid during the assay which is described in further detail herein. The portion of the sample pad 14 that is folded over the distal end of the backing card 24 is referred to as an end portion 20 of the test strip 10. The distal portion of the sample pad 14 that is downstream of the folded end portion 20 and on the front surface of the test strip 10 is the filter membrane 18, which receives a sample 50 during the assay as described in further detail herein.

The backing card 24 provides rigidity to the test strip 10 and is coated with an adhesive to hold the various membranes in place. The backing card 24 can be made from various materials including, but not limited to, polystyrene or PVC, for example. Other materials may also be used for the backing card 24 as is known by those skilled in the art.

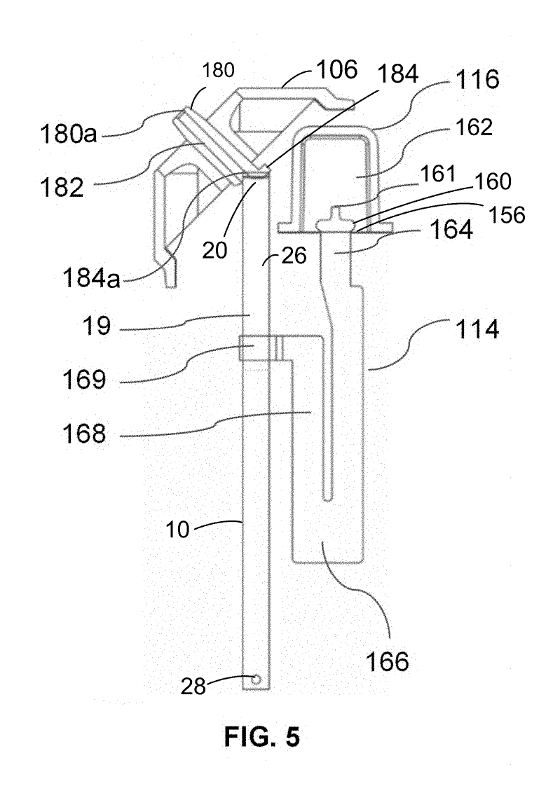

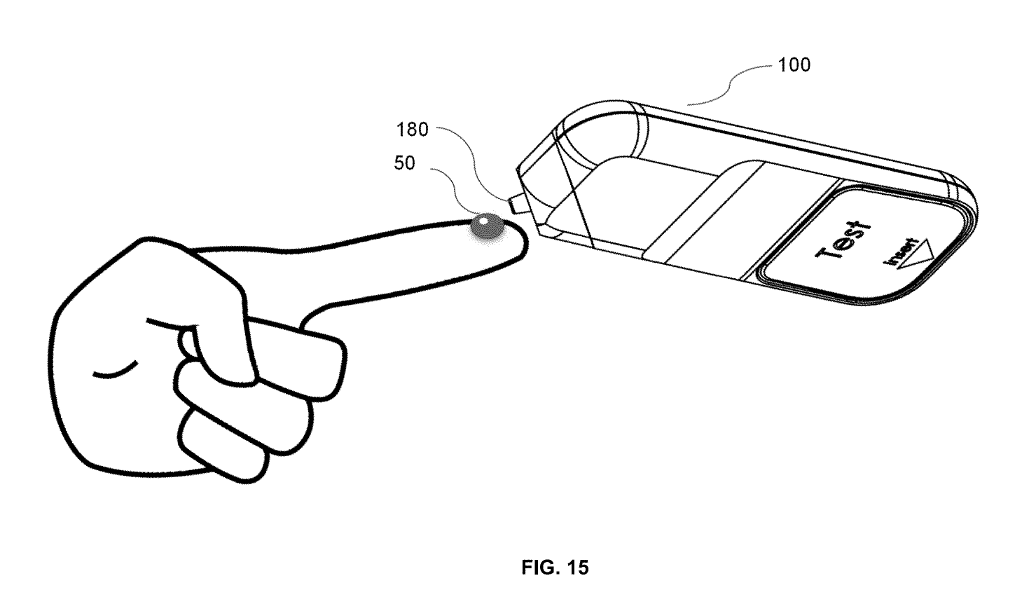

The filter membrane 18, which is part of the sample pad 14, filters sample analytes from the sample 50, such as blood cells from a blood sample (see FIG. 15) leaving plasma to flow freely into the other membranes of the test strip 10 without clogging the other membranes or interfering with the assay. In various embodiments, the sample 50 being tested can be blood, saliva, semen, tears, beverages, or other liquid solutions. The filter membrane 18 is also very absorptive such that it can wick a portion of the blood sample 50 out from a capillary tube exit 184 (see FIG. 5) when a cartridge that contains the test strip 10 is actuated to begin the assay (e.g. placed within an electronic reader 250) in accordance with the teachings herein.

The conjugate pad 16 contains various chemicals that are used for performing the assay where these chemicals include, but are not limited to, buffers, surfactants, salts, antibodies and labelling agents. The particular chemicals that are used depend on the type of test that is being done (i.e. which analytes are being tested for in the sample 50).

The reaction membrane 21 is typically made from nitrocellulose. The reaction zones 22 can be made by immobilizing a capture agent or an antigen in a small area on the reaction membrane 21. The reaction zones 22 are typically rectangular zones that are perpendicular (i.e. transverse) to the length (i.e. longitudinal direction or longitudinal axis) of the strip 10. The reaction zones 22 are areas that are measured to determine the result of the reaction (i.e. the result of the assay/test). One of the reaction zones 22 is used for the detection of the biomarker in question (and there is one reaction zone per biomarker being detected), and the other of the reaction zones 22 is used as a control. One of the reaction zones 22 is used as a control to ensure that the test cartridge did not malfunction, and also for quality control purposes. In alternative embodiments, there may be more than two reaction zones, which may occur where more than one biomarker is detected on a single test strip; this is known as multiplexing. In these embodiments, there is one reaction zone for each biomarker that is to be detected and an additional reaction zone that is used as a control.

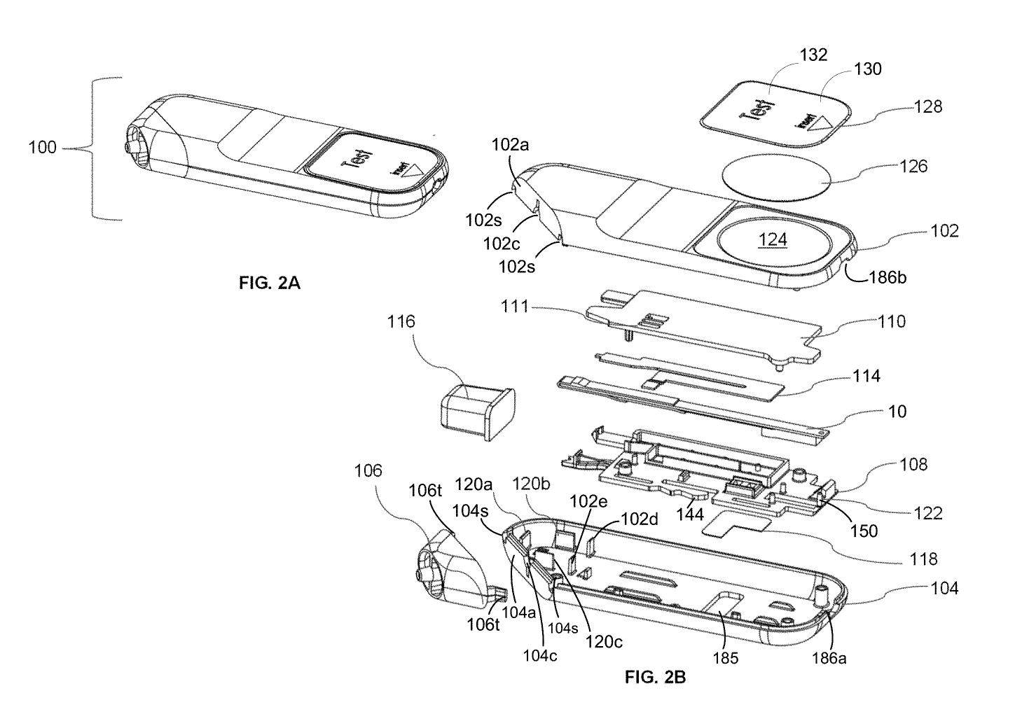

The absorbent pad 12 is large and dense to absorb a sufficient amount of the sample 50 and the buffer liquid 162 that can be used to properly complete the assay. The absorbent pad 12 has a maximum absorbance capacity, and a change in the volume of the sample 50 or the buffer liquid 162 would require a change in the dimensions of absorbent pad 12. The aperture 28 may be cut into the backing card 24 to provide accurate positioning when the test strip 10 is placed into a test strip holder (see FIG. 2B) of a cartridge 100 (see FIG. 2A) in accordance with the teachings herein.

In this example embodiment, the sample pad 14 is folded such that it brings certain liquids, such as a buffer liquid 162 from the buffer entrance area 19 on the rear surface of the test strip 10 to the other side of the test strip 10 where the conjugate pad 16 is located. The folding of the filter membrane 18 has many advantages. The folded filter membrane 18 allows the test strip 10 to be shorter and thereby allowing it to fit in a more ergonomic and smaller sized cartridge. The folded filter membrane 18 allows the sample 50, which in this example is blood, to enter the strip 10 at the end portion 20 of the test strip 10 while the buffer liquid enters on the same filter membrane 18 on the back surface of the test strip 10. This allows a capillary assembly 106, which is used to receive the sample 50, to be positioned at the end of the cartridge 100 where it will be in close proximity to the end portion 20 of the test strip 10. The location of the capillary assembly 106 at the end of the cartridge improves the user experience during sample collection.

The sample (e.g. blood) 50 enters the top end portion 20 of the test strip 10 and soaks into the filter membrane 18. The blood resuspends and mixes with chemicals that were dried onto the filter membrane 18 during manufacture. These chemicals include at least one of pH buffering agents and surfactants to prepare the sample 50 for the detection reaction. The mixture of chemicals is chosen to match the type of sample 50 and may change depending on the biomarker being detected. For instance, the chemicals may include reagents to remove components from the sample 50 that may interfere with the detection reactions. The blood cells are trapped within the fiber matrix of the filter membrane 18.

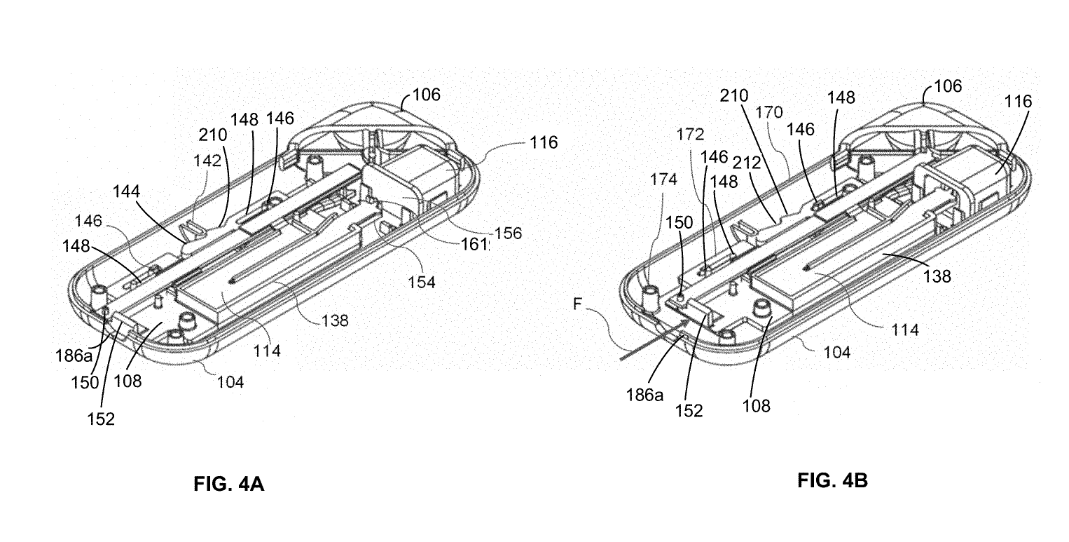

After a portion of a strip holder punctures a buffer capsule 116, the buffer liquid 162 enters a buffer channel pad 114 (see FIGS. 4A-4B and 5). The buffer liquid 162 then soaks into the buffer channel pad 114 and travels toward the test strip 10. The travel time of the buffer liquid 162 is such that the blood sample 50 is able to completely soak into the filter membrane 18 before the buffer liquid 162 enters the test strip 10. This ensures proper filtration of the blood cells. As the buffer liquid 162 flows into the buffer entrance area 19 at the back of the test strip 10, the buffer liquid 162 flows into and reaches the end portion 20 of the strip 10, where it will meet the blood sample 50. This lets the blood sample 50 and the buffer liquid 162 to wick through the rest of the test strip 10, effectively “pushing” the sample 50 through the proximal portions of the test strip 10, thereby performing the assay. The blood cells are left behind in the filter membrane 18 of the sample pad 14 so they do not interfere with the rest of the assay.

The blood plasma of the sample 50 continues along to the next membrane, which is the conjugate pad 16, where the blood plasma of the sample 50 picks up additional chemicals needed to perform the assay. The blood plasma then wicks into the reaction membrane 21 where it travels to the reaction zones 22. As the blood plasma travels down the reaction membrane 21 the blood plasma mixes and reacts with reagents resuspended therein from the conjugate pad 16. When the blood plasma reaches the reaction zones 22, the label in blood plasma solution selectively binds to the reagent that is located at the reaction zones 22, thereby immobilizing the blood plasma. Unbound antigens and label reagent continue to flow through the reaction membrane 21 into the absorbent pad 12.

A device, referred to herein as electronic reader 250, then measures the concentration of the label remaining at the reaction zones 22. The amount of the label can be measured by a color measurement, fluorescence measurement or other indicator depending on the type of label used. The amount of label remaining is correlated to the concentration of the target antigen in the blood sample 50 thereby providing the results of the assay.

There are many different formats of immunoassays that are compatible with the assay format provided by the test strip 10. These immunoassays include, but are not limited to, a competitive assay format in which an antibody is deposited at the reaction zones 22, a competitive assay format in which the competitive agent is deposited at the reaction zones 22, and a non-competitive sandwich assay format.

An example embodiment of a cartridge 100 is shown in FIGS. 2A-9C. FIG. 2A shows an isometric view of the cartridge 100 and FIG. 2B shows an isometric exploded view of the cartridge 100.

The assay components are enclosed within first and second cartridge enclosure pieces 102 and 104. The test strip 10 is held in place by first and second strip holder pieces 108 and 110. A buffer capsule 116 is positioned at the top of the cartridge 100 and is held firmly in place by ribs 120a-120e on the cartridge enclosure piece 104 as well as an opposing surface wall of enclosure piece 102. The buffer capsule 116 includes a buffer reservoir (physical piece), the buffer liquid 162 and a seal 156 (e.g. a foil covering). The capillary assembly 106 has tabs 106t that engage slots 104s on an angled upper surface wall 104a of the enclosure piece 104 and slots 102s on an angled upper surface wall 102a of the enclosure piece 102 so that the capillary assembly 106 is located at an upper portion of the cartridge 100. This position of the capillary assembly 106 allows the sample 50 to more easily enter the cartridge 100 and flow to the strip 10. The slots 106t of the capillary assembly 106 are sandwiched between opposing portions of the enclosure pieces 102 and 104. An NFC tag 126 may be attached to the enclosure piece 102 and is covered with a cover 128. In an example embodiment, the NFC tag 126 may be embedded in the cover 128. It should be noted that the NFC tag 126 can also be called an NFC chip.

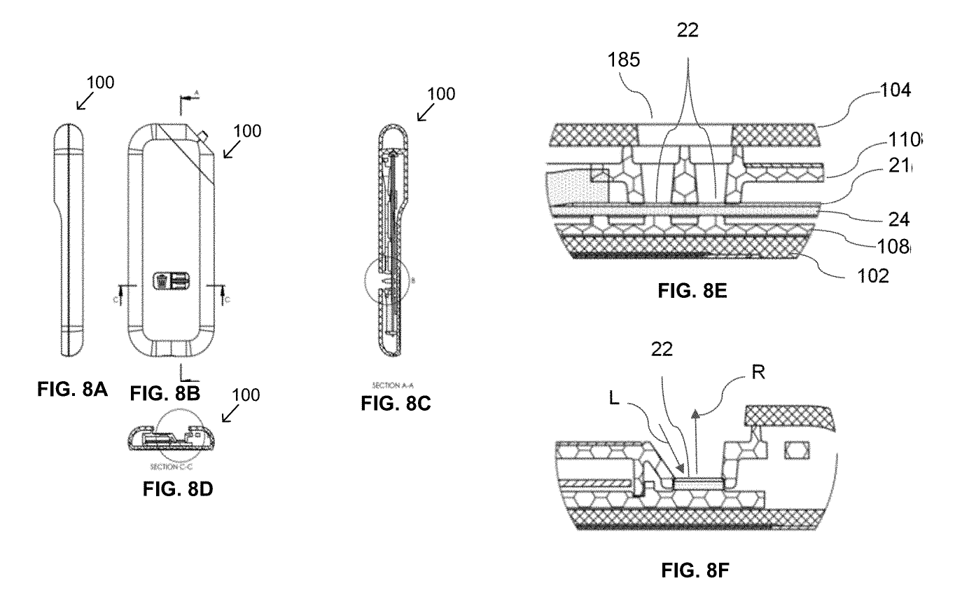

The first and second cartridge enclosure pieces 102 and 104 provide a housing for the cartridge 100 to shield and protect the test strip 10 and the assay system from outside elements. This prevents user tampering, as well as dust or dirt from entering the optical area (see FIGS. 8C-8F) of the cartridge 100, which may affect the chemistry of color measurement. The first and second cartridge pieces 102 and 104 also limit the effect of humidity on the various membranes of the test strip 10 thereby improving performance and lengthening the amount of time that the cartridge 100 can be used after being removed from the packaging. The first and second cartridge enclosure pieces 102 and 104 may be made of plastic.

The rectangular format/shape of the cartridge 100 allows the user to hold the cartridge 100 comfortably when collecting the sample 50 and when inserting the cartridge 100 into the electronic reader 250. The cover 128 includes a test indicator 132 that allows the user to identify the type of cartridge 100 and therefore the type of test that may be performed with the cartridge 100. The test indicator 132 may be a sticker.

The cartridge enclosure has an opening 185 that reveals the reaction zones 22 to the optical system 234 of the electronic reader 250 when the cartridge 100 is inserted into the electronic reader 250. The enclosure of the cartridge 100 also has a small aperture 186 in its bottom end formed by notch 186a of cartridge piece 102 and notch 186b of cartridge piece 104. The aperture 186 may be referred to as an actuation aperture. A combination of ribs 146 and pegs 174 hold the rest of the components of the cartridge 100 in place. The first and second cartridge enclosure pieces 102 and 104 are aligned by the pegs 174 and may be welded together at the edges 170 using appropriate plastic welding techniques such as ultrasonic welding for example.

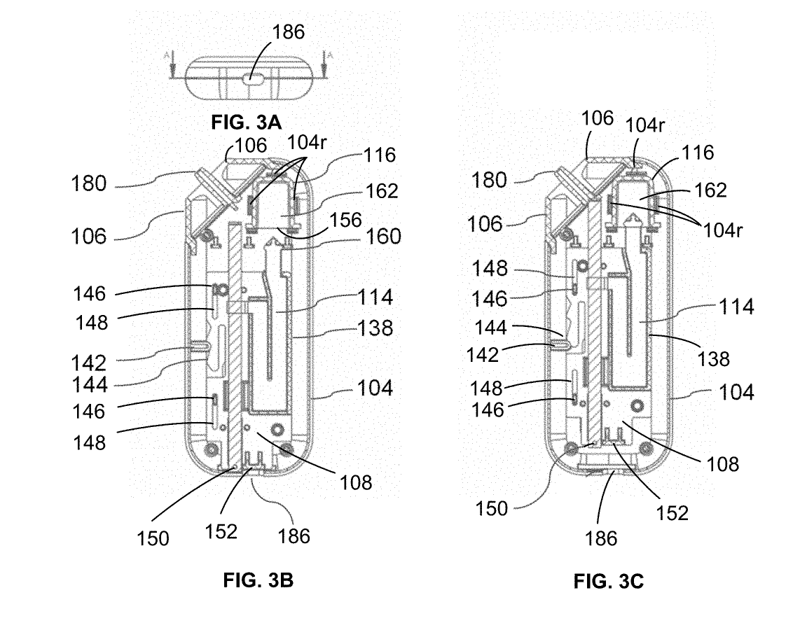

Referring now to FIGS. 2B-6D, the strip holder has two pieces 108 and 110 that sandwich the test strip 10 and the buffer channel pad 114 holding them in place. The buffer channel pad 114 may also be referred to as a buffer channel membrane. The test strip 10 is aligned with a pin 150 that is located at one end of the strip holder piece 108 such that the pin 150 goes through the aperture 28 to hold the test strip 10 in place. The strip holder is held in place by a sliding rail system 172 which allows the strip holder to move during use. The sliding rail system 172 comprises slide pins 146 that fit into and move along slots 148 on the enclosure piece 104 which allows the strip holder to move during use. Accordingly, the strip holder has a pre-actuation position as shown in FIGS. 3B and 4A where the end of the test strip 10 having the absorbent pad 12 is positioned at a bottom edge of the cartridge 100.

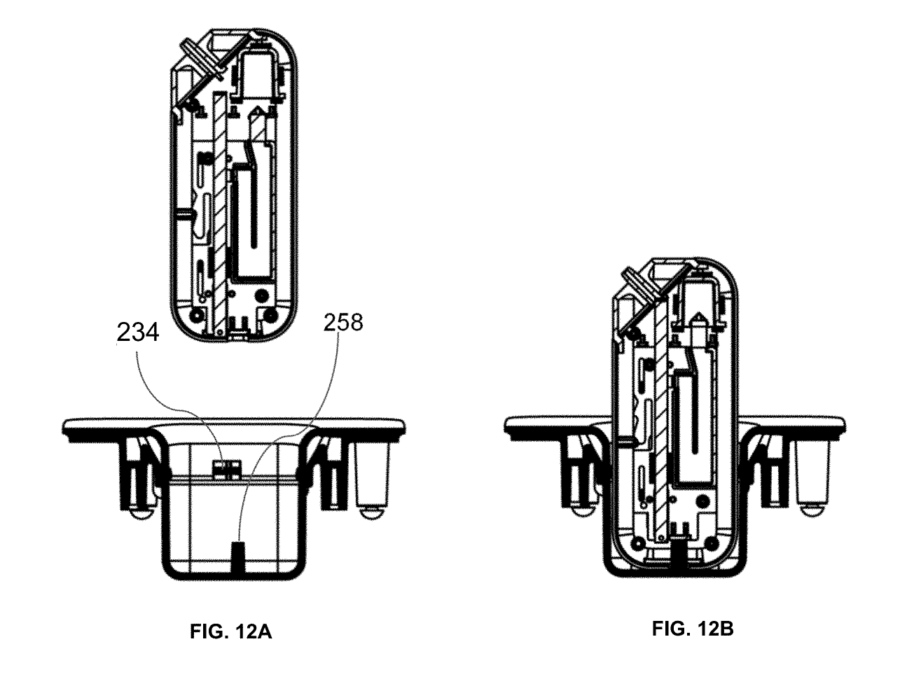

When actuation is performed to start the assay, a force F (see FIG. 4B) is applied to the bottom of the strip holder which causes the strip holder to be actuated to a post-actuation position as shown in FIGS. 3C and 4B. The aperture 186 provided by the notches 186a and 186b in the enclosure pieces 104 and 102 at the bottom edge of the cartridge 100 allows the force F to be applied therethrough. For example, the electronic reader 250 comprises a tab 258 (see FIG. 12A) that applies the force F against a wall or flange 152 on the strip holder. Therefore, the strip holder moves within the enclosure of the cartridge 100 when the cartridge 100 is inserted into the electronic reader 250.

The cartridge 100 contains a buffer capsule 116, which is filled with a buffer solution 162 during manufacture. The buffer capsule 116 is located adjacent to the capillary tube 182. An end portion 164 of the buffer channel membrane (i.e. buffer channel pad 114) that is nearest to the buffer capsule 116 includes a fang or protrusion 160 (see FIG. 4A). The first function or result of the movement of the strip holder during actuation to the post-actuation position is that the pointed end 161 of the fang 160 moves towards the buffer capsule 116 to puncture a bottom side of the buffer capsule 116 which releases the buffer fluid 162 that is contained within the buffer capsule 116. The buffer fluid 162 begins to flow downwards towards and along the buffer channel membrane 114 and accumulates in the lower part 166 of the buffer channel membrane 114 (see FIG. 5).

The second function or result of the actuation of the strip holder is to bring an end of the test strip 10 into contact with a portion of a capillary assembly 106 to receive the sample 50. The capillary assembly 106 includes a first end 180 having a first aperture 180a, a second end 184 having a second aperture 184a and a capillary channel or capillary tube 182 from the aperture 180a of the first capillary end 180 to the aperture 184a of the second capillary end 184. The aperture 180a of the first capillary end 180 is open to the outer environment for receiving the sample 50 while the aperture 184a of the second capillary end 184 is disposed within the cartridge 100 for bringing the sample 50 to the end portion 20 of the test strip 10. This allows the sample 50 to move from the capillary tube 182 to the test strip 10 during use.

When both of the first and second results happen in quick succession, the timing is such that the sample 50, e.g. blood, fully enters the test strip 10 from the capillary tube 182 before the buffer liquid 162 passes from the buffer capsule 116 through the various portions 164, 166, 168 and 169 (see FIG. 5) of the buffer channel membrane 114 onto a portion (i.e. buffer entrance area 19) of the test strip 10.

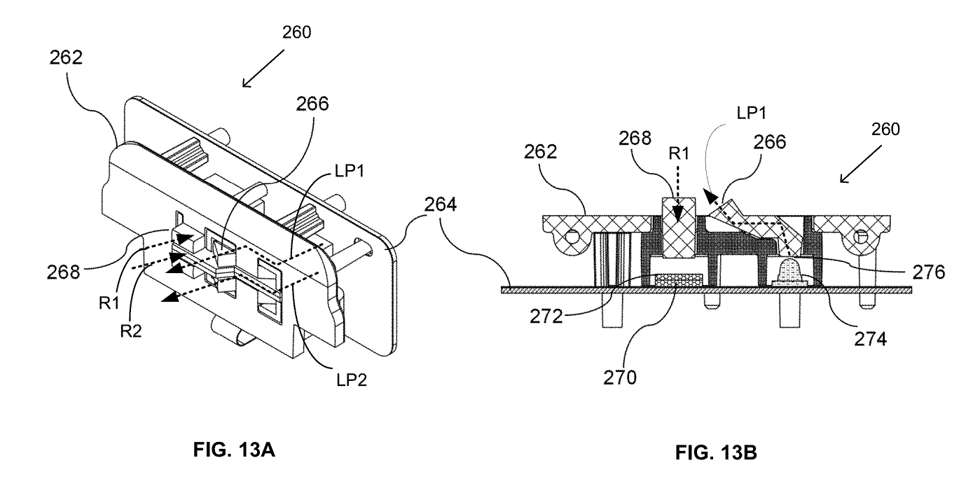

The third function or result of the actuation of the strip holder is to bring the reaction area 22 of the test strip 10 into alignment with an opening 185 in the enclosure piece 104 and therefore in alignment with various optical components (see FIGS. 12A-13B) of an optical system 234 of the electronic reader 250.

Referring now to FIGS. 3B, 4A, 4B, 6C-6D, and 19C, a detent 144 is formed at a portion of the strip holder piece 108. The detent 144 comprises an arm 210 that is a flexible piece of plastic of the strip holder piece 108. The detent 144 also comprises a notch 212 and a curved end portion 214. The notch 212 or the curved end portion 214 of the detent 144 is engaged by a peg or boss 142 that is on an inner surface of the enclosure piece 104 depending on whether the strip holder is in the pre-actuation or post-actuation position. The engagement of the notch 212 or the end portion 214 of the detent 144 with the boss 142 provides the user with tactile feedback so that the user knows when the cartridge 100 is inserted properly into the electronic reader 250. This is because when the strip holder moves during insertion of the cartridge 100 into the electronic reader 250, the peg 142 moves from engaging the notch 212 of the detent piece 144 to engaging the end portion 214 of the detent 144. The detent 144 also prevents the cartridge 100 from accidental partial actuation or slow actuation, which may cause test failure. Rather, the interaction between the detent 144 and the peg 142 ensures that both the transfer of the sample 50 along the capillary tube 182 to the test strip 10 and the puncture of the buffer capsule 116 happen approximately simultaneously. The force profile for the detent 144 is such that it snaps into place after a certain amount of force F is applied to the bottom flange 152 of the strip holder.

The profile of the detent 144 can be tuned by changing its geometry (e.g. width, angle, length, etc.) to accommodate different desired forces. Accordingly, the detent 144 and the peg 142 will require a certain amount of force to move the strip holder that a user may generally be able to provide, but the required force will be large enough that it prevents accidental or unwanted movement of the strip holder during storage and transportation of the cartridge 100. This prevents rattling of the strip holder pieces 108 and 110 and unintended puncture of the buffer capsule 116.

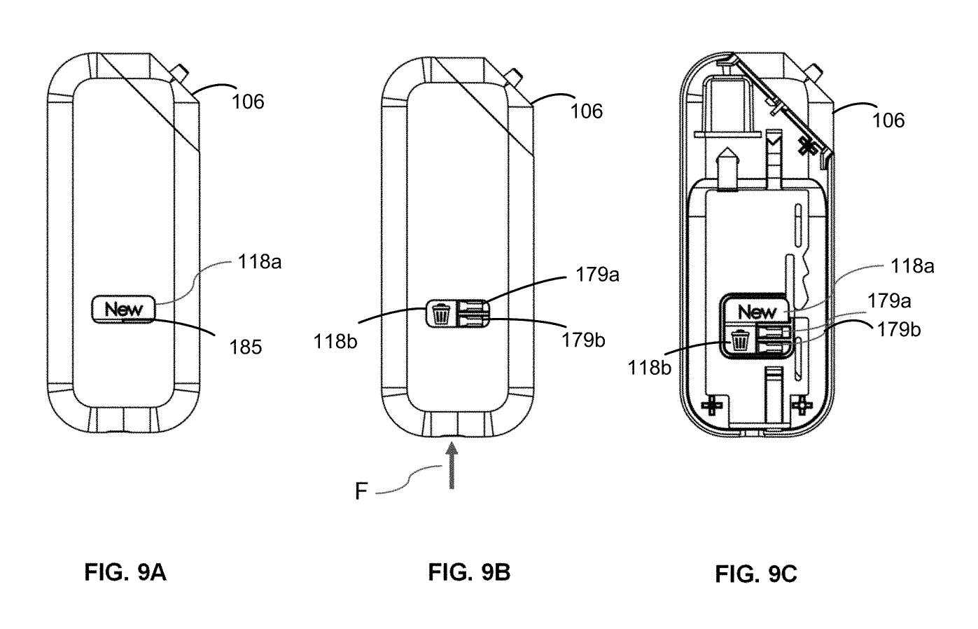

Referring now to FIGS. 2B and 9A-9C, the cartridge 100 comprises a status indicator 118 which indicates whether or not the cartridge 100 has been used. For example, when the strip holder is in the pre-actuation position, the cartridge 100 has not been used and the status indicator 118 is visible through the opening 185 in the enclosure piece 104. The status indicator 118 may have a certain symbol, color, word or picture to indicate that the cartridge 100 has not been used. In this example, embodiment, the status indicator 118 includes a first indicator an upper portion 118a having the word “NEW” which is visible through the window 185 when the strip holder is in the pre-actuation position and therefore indicates that the cartridge 100 has not been used. Once the strip holder has been actuated and is in the post-actuation position, the cartridge 100 performs and finishes the assay. The position and therefore portion of the status indicator 118 that is visible in the window 185 changes such that when the strip holder is in the post-actuation position the bottom portion 118b of the status indicator 118 having a second indicator (e.g. a different label) is visible through the window 185 to indicate that the cartridge 100 is used. For example, the lower portion 118b of the status indicator 118 can include the word “USED” or have an appropriate image such as a garbage can, for example. The actuation of the strip holder to the post-actuation position is irreversible in that the detent 144 and the peg 142 prevent the user from accidentally moving the strip holder from the post-actuation position back to the pre-actuation position thereby preventing the reuse of the cartridge 100.

In an alternative embodiment, the status indicator 118 can also include color changing materials that indicate that the cartridge 100 has been exposed to prohibited humidity or temperature during storage such that the cartridge 100 may not provide accurate test results. For example, the status indicator 118 may change color indicating that the cartridge 100 can no longer be used to provide a valid assay.

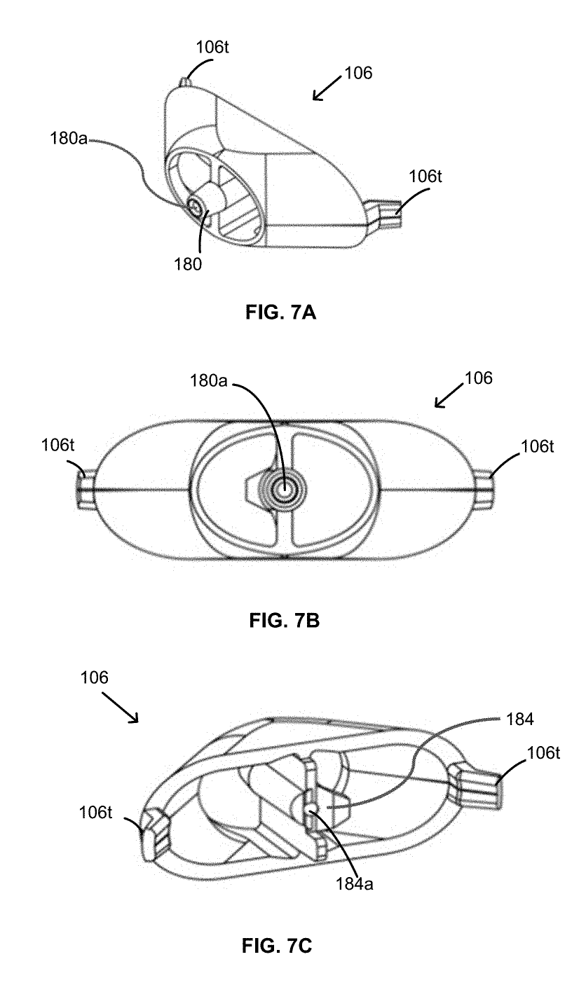

The capillary assembly 106 having the capillary tube 182 is shown isolated in FIGS. 7A-7C. The capillary tube 182 is used to control the amount of sample 50 that enters the test strip 10. The length and width of the capillary tube 182 is chosen based on the desired amount of sample 50 that is used to perform the assay. Capillary action wicks the sample 50, such as blood or another liquid, for example, from the aperture 180a of the outer facing end 180 of the capillary assembly 106 into the capillary tube 182. Once the capillary tube 182 is full, no more sample 50 will be wicked into the capillary tube 182. The sample 50 in the capillary tube 182 will be transferred to the test strip 10 when the strip holder is actuated to the post-actuation position at which time the end portion 20 of the test strip 10 will contact with the aperture 184a of the inner facing end 184 of the capillary assembly 106. The capillary tube 182 can be made using glass or plastic or it may be integrated into one of the cartridge pieces 102 or 104.

In a different embodiment, another method for implementing the transfer of sample 50 to the test strip 10 is to move the capillary tube 182 to the end portion 20 of the test strip 10 while keeping the test strip 10 stationary. When the end portion 20 of the test strip and the end 184 of the capillary tube 182 touch, the sample 50 in the capillary tube 182 will wick out into the filter membrane 18 of the test strip 10. The sample 50 contained in the capillary tube 182 is then transferred to the filter membrane 18 of the sample pad 14. This transfer also happens due to capillary action. This is advantageous due to combining several steps of the assay together so that they happen automatically for the user, and also for perfectly timing the entrance of the sample 50 to the test strip 10 followed by the entrance of the buffer liquid 162 into the test strip 10, thereby increasing the consistency of the test.

The entrance end 180 of the capillary tube 182 is the end in which the sample 50 is collected and can be tapered or rounded to allow the sample 50 to more easily enter into the capillary tube 182. The capillary tube 182 may be treated via a plasma or corona treatment, surfactant or other surface treatment to ensure high surface energy and good wetting ability for capillary action.

The capillary assembly 106 may be made from a transparent material to allow the user to see the level of sample 50 that is in the passage of the capillary tube 182. The entrance end 180 of the capillary tube 182 is positioned at an upper corner of the capillary assembly 106 in such a way that the end 180 of the capillary tube 182 aligns with an upper corner/edge of the capillary assembly 106, so that the user can see when the capillary tube 182 is full which occurs when the sample 50 has filled the visible area. The visibility provided by the transparent capillary assembly 106 also allows the user to see the sample 50 leave the passage of the capillary tube 182 as it enters into the test strip 10, thereby confirming that the test/assay has begun.

Advantageously this creates a failsafe for incorrect usage. If too little sample 50 is inserted into the capillary tube 182, none of the sample 50 will transfer to the test strip 10. Inaccurate test results occur if the wrong sample volume is used. In accordance with the teachings herein, the mistake of insufficient sample volume is noticeable to both the user through the transparent capillary assembly 106 and the electronic reader 250 due to lack of a control signal when performing optical readings as discussed in further detail below. The control signal is an optical signal that is measured by the electronic reader 250 from one of the reaction zones 22 that acts as a control to ensure that the test strip 10 is operating properly.

The test strip 10 is moved into place during actuation of the strip holder such that the end portion 20 of the test strip 10 touches the exit aperture 184a of the capillary tube 182. The test strip 10 may be actuated/moved such that the end portion 20 of the test strip 10 slightly extends into the exit aperture 184a and into the passage/channel of the capillary tube 182. This ensures proper transfer of the sample 50 from the capillary tube 182 to the test strip 10.

Referring now to FIGS. 3B-3C and 5, the buffer membrane 114 is surrounded by buffer storage area walls 138 that are part of the strip holder 124. The buffer storage area walls 138 form a waterproof chamber to hold any excess liquid that may pool in the bottom section 166 of the buffer membrane 114 due to gravity, and remain sealed regardless of the orientation of the cartridge 100.

The buffer membrane 114 has an intake area 164 that is sandwiched between adjacent sides of the strip holder pieces 108 and 110 and is downstream of the fang 160 to drain the buffer liquid 162 from the buffer capsule 116 so that it quickly soaks into the rest of the dry buffer membrane 114 due to capillary wicking and gravity. Excess buffer liquid 162 pools at the bottom of the buffer membrane 166 due to gravity. This allows the buffer liquid 162 to be drawn out of the buffer membrane 114 in a consistent manner through the buffer membrane exit 169 which includes a small arm, the size of which can be tuned to determine the rate and timing at which the buffer liquid 162 flows into the test strip 10. The buffer liquid 162 leaves the buffer membrane 114 and flows into the test strip 10 at the buffer entrance area 19 (of the sample pad 14) on the test strip 10. The buffer liquid 162 is continuously drained through the test strip 10 from the excess buffer area 166 and region 168 of the buffer membrane 114.

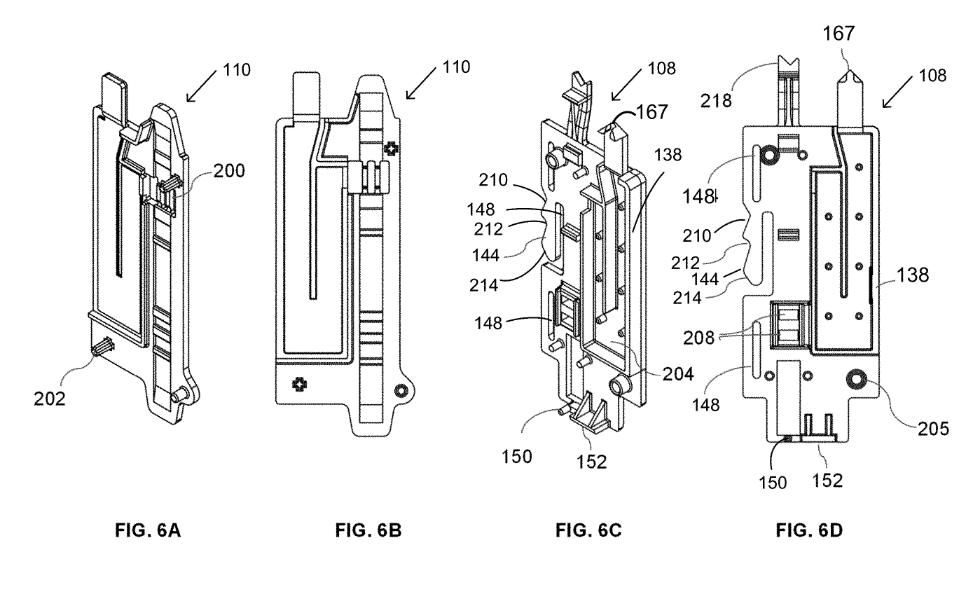

The buffer membrane 114 stores the buffer liquid 162 that is emptied from the buffer capsule 116. In this example embodiment, the buffer membrane 114 is not attached to the test strip 10 through an adhesive, but rather may be physically pressed in contact with the back surface 26 of the membrane strip 14 at the buffer entrance area 19 by ridges on the strip holder 200 (see FIG. 6A). This helps in providing an even flow of buffer liquid 162 into the test strip 10. The buffer membrane exit 169 is a narrow arm that touches the test strip 10 and also delays the flow of the buffer liquid 162 to the test strip 10. The shape, size and material of the buffer membrane exit 169 can be changed to tune the time that it takes for the buffer liquid 162 to completely flow into the test strip 10. This ensures efficient emptying of the buffer capsule 116 and consistent entry of the buffer liquid 162 into the test strip 10.

The buffer liquid 162 is stored in the buffer capsule 116 to be used when the test is performed. During use, the buffer liquid solution 162 moves into the test strip 10, mixes with the sample 50 and pushes the sample 50 along the test strip 10. Three components that allow for these actions include the buffer liquid capsule 116 (which can also be called the buffer liquid storage container), the fang 160 (which can also be called the puncture mechanism 160) with the sharp end or point 161, and the buffer membrane 114.

Initially, the buffer capsule 116 has an opening on one side. During manufacturing, the liquid buffer 162 is placed into the buffer capsule 116 through this opening after which the opening of the buffer capsule 116 is sealed by a film 156. For example, the opening can be sealed with a plastic or metal covering, or via ultrasonic welding or heat sealing. An advantage of this structure is that it facilitates easy filling of the buffer liquid 162 into the buffer capsule 116 and then sealing of the buffer capsule 116. The buffer capsule 116 may be formed using various techniques including, but not limited to, heat forming, injection molding or other appropriate methods as is known by those skilled in the art, for example. In this example embodiment, the buffer capsule 116 is made from injection molded, non-leaching plastic.

In this example embodiment, the buffer capsule 116 is held firmly in place in the cartridge 100 by ribs 104r that protrude from an inner surface of the enclosure piece 104 and the buffer capsule 116 is also sandwiched between opposing inner surfaces of the enclosure pieces 102 and 104.

In this example embodiment, the buffer liquid 162 in the buffer capsule 116 may be released by puncturing the foil film (i.e. seal) 156, which is used to seal the buffer capsule 116. The act of puncturing may be performed when the user of the cartridge 100 or when a machine applies a force through mechanical movement or pressure from the user or an element of the machine such as a moveable tab. For example, a piece of plastic that is shaped to a point, which is referred to herein as the buffer fang 160 with the point 161, is positioned in-line with and spaced apart from the buffer capsule 116 so not physical contact is made in the pre-actuation position, which occurs before the cartridge 100 is used to perform an assay. However, during use, when the cartridge 100 transitions from the pre-actuation position to the post-actuation position, the fang 160 enters the buffer capsule 116 thereby releasing the buffer liquid 162 which then quickly soaks into the buffer membrane 114.

The buffer capsule 116 is inverted at the top of the test cartridge 100 and oriented in a vertical manner during use to enable full transfer of the buffer liquid 162 into the buffer membrane 114. The buffer membrane 114 ensures efficient use of the buffer liquid 162, improving consistency and decreasing the volume required compared to using a dropper system. In a traditional lateral flow assay, the buffer liquid 162 is added via a dropper into the system. This is inconvenient for the user and opens the system up to additional errors. For example, with a conventional dropper system, the size of a drop can vary in volume or be miscounted. Furthermore, with a conventional dropper system, the drops may miss the intended opening leaking into other areas, the drops may have different volumes, the user may apply the wrong number of drops, or the user may add the drops at different times leading to inconsistency in testing. The “free flowing” liquid that occurs with a conventional dropper system also has an additional risk of pooling or rolling into a part of the test interfering with the test operation. In contrast, with the test strip system described herein, a fixed volume of buffer liquid 162 is advantageously dispensed into the buffer capsule 116 for storage during manufacturing by a precise dispensing system. In addition, the use of the buffer membrane 114 and the fang 160 ensures that all or most of the buffer liquid 162 in the buffer capsule 116 is sucked out while the capillary action of the buffer membrane 114 provides a consistent release of the buffer liquid 162 into the test strip 10.

The buffer fang 160 is shown in FIGS. 4A-5 and 6C-6D. A gap 167 in the center of the fang 160 allows the buffer liquid 162 to flow out into the tip 164 of the buffer membrane 114 at any puncture depth, so that even a short puncture distance will cause the buffer liquid 162 to be sucked out of the buffer capsule 116 by capillary action. The buffer fang 160 sandwiches the end of the buffer membrane 112 so that is able to wick the buffer liquid 162 out into the buffer storage area at the bottom of the buffer membrane 166.

The buffer membrane 114 can contain dried chemicals such as pH-altering agents or surfactants that are dissolved as the buffer liquid 162 from the buffer capsule 116 flows through the buffer membrane 114. It may not be desirable to have these chemicals dissolved in the buffer capsule 116 as they may have a short shelf life in liquid form compared to a solid state, they may interfere with the sealing process for the buffer capsule 116, and/or they may react with the plastic that is used to form the walls of the buffer capsule 116.

The shape and size of the buffer membrane 114 determines the transit time which is the amount of time that it takes for the buffer liquid 162 to enter the test strip 10. For example, the buffer membrane 114 may be dimensioned (i.e. a certain length and width are selected) to obtain a delay time of 30 seconds. The geometry, such as the length of fluid path and the cross-sectional area determine the delay time. The internal structure of the buffer membrane 114 and/or the surface treatments may also be varied to speed up or slow down the delay time. For example, the buffer membrane 114 may be chosen to be a loose and low-retention membrane, with a low capillary pressure so that the test strip 10, with a higher capillary pressure (i.e. suction), is able to pull the buffer liquid 162 from the buffer membrane 114. For example, Millipore G041 may be used as the buffer membrane 114. Alternatively, in other embodiments, the buffer membrane 114 may comprise other conjugate release pads and sample pads such as, but not limited to, for example, those from GE Healthcare, such as Standard 14 and MF1, respectively, and Ahlstrom-Munksjö, such as 6613 and 8950.

The buffer liquid 162 leaves the buffer membrane 114 and enters the test strip 10 at the junction 169 (see FIG. 5). The area where the sample buffer pad 18 contacts the junction 168 has the plastic cut away in region 111 (see FIG. 2B) of the strip holder piece 110. This inhibits the buffer liquid's ability to form a drop and pool at this location.

In addition, areas 170 (see FIG. 4B) of the outer enclosure piece 104 of the cartridge 100 and areas 138 of the strip holder may be ultrasonically welded to hold these elements in place. These areas may also be glued or rely on a tight snap fit to provide a waterproofing fit. This both serves to contain liquid in the intended areas and provides an additional barrier if the cartridge 100 malfunctions, so that liquid does not leak outside into the external reader 250 or onto the user.

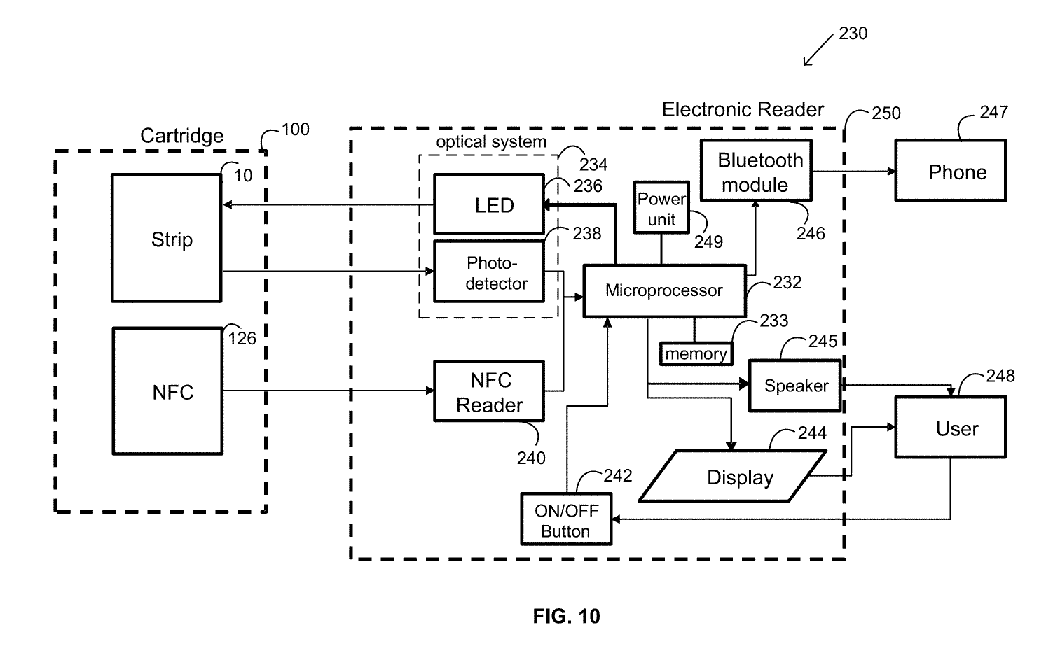

Referring now to FIG. 10, shown therein is an example embodiment of a test kit 230 that comprises a cartridge 100 for performing an assay and an electronic reader 250 for measuring the test results. The cartridge 100 includes a test strip 10 and an NFC 126 as previously described. The electronic reader 250 comprises a microprocessor 232, a memory 233, an optical system 234 including a light source 236 (e.g. LED) and a photo-detector 238, an NFC reader 240, an on/off button 242, a display 244, and a power unit 249. It should be understood that the electronic reader 232 may include other components that are needed for operation that are not shown in FIG. 10 but are understood by those skilled in the art. For example the electronic reader 232 can also include an analog to digital convertor, and a digital to analog convertor (this may not be needed in cases where components already include a digital to analog conversion mechanism) (all not shown). The optical system 234 can also include further optical components such as one or more filters or light guides as is described in further detail with respect to FIGS. 13A-13B. Furthermore, it should be understood that in some embodiments some components of the electronic reader 250 are optional since they may not be used in certain cases. For example, in some embodiments, the cartridge 100 does not include the NFC 126 in which case the electronic reader 250 does not have the NFC reader 240.

The microprocessor 232 controls the operation of the electronic reader 250 and includes memory for storing operational parameters and certain data that is collected. The microprocessor 232 begins operation when it receives an operational signal from the actuation of the on/off button 242 when a user 248 turns the electronic reader 250 on or off. When the operational signal indicates that the electronic reader 250 is to turn on, the microprocessor 232 can initialize the operation of the other components of the electronic reader 250 and optionally run certain diagnostic tests to ensure that the components of the electronic reader 250 are working properly. The microprocessor 232 then remains in a “wake” or “alert” state until the user 248 begins an assay by inserting the cartridge 100 into the electronic reader 250. The microprocessor 232 may be a standard processor, such as an Intel processor or an ARM processor, for example.

When the cartridge 100 is inserted into the electronic reader 250, the microprocessor 232 can poll the NFC reader 240 to scan for the NFC 126 to determine the type of test that will be performed with the cartridge 100. The microprocessor 232 can then determine certain test parameters based on the type of test that can be performed by the cartridge 100. For example, the test parameters can include the timing that is required for measuring the test results after the test has begun, as well as the control parameters needed to properly operate the optical system 234 to measure the test results.

The microprocessor 232 can send display data to the display 244, which can then output the display data for the user 248 to view. The display data may include status or operational data to indicate the operational status of the electronic reader 250, test data including the test results and error data to indicate whether any operational errors have occurred during the testing. In some embodiments, the display 244 can be touch sensitive so that the user 248 can enter control information for controlling the operation of a test or other data, such as test data that can indicate the type of test being performed and/or the name of the individual from whom the sample 50 was obtained. The display 244 may be, but is not limited to, an OLED or LCD display and in some cases may be a touch screen such as that used in tablets or smart phone devices. For example, the microprocessor 232, through executing certain programs such as a graphical user interface (GUI) engine, can generate a user interface which is then show on the display 244. The GUI engine provides data according to a certain layout for each user interface and also receives data input or control inputs from the user 248. The GUI then uses the inputs from the user to change the data that is shown on the current user interface, or changes the operation of the electronic reader 250.

The microprocessor 232 can also send audio control data to the speaker 245 for generating certain sounds to provide information to the user 248 during the test. For example, the audio control data may be used to generate test status sounds to indicate the status of a test to the user 248 or error sounds to indicate when an error occurs during operation.

The microprocessor 232 can also communicate with other electronic devices to provide or receive information by sending and receiving data to the Bluetooth module 246. For example, the microprocessor 232 can use the Bluetooth module 246 to communicate with the user's cell phone 247. For example, the microprocessor 232 may send test result data to the user's cell phone 247 or send error codes to the user's cell phone 247 if errors occurred during operation. The cell phone 247 may be a smart phone. Other electronic devices may be used instead of the phone 247 as is commonly known by those skilled in the art. An example of another electronic device would be a connected hub or wireless printer.

The power unit 249 can include any suitable power source along with a voltage convertor and other circuitry needed to generate a power signal that can be used to provide power to the various components of the electronic reader 250. For example, power unit 249 may include a power adaptor for connection to a power line through an electrical outlet. Alternatively, the power unit 249 may be connectable to one or more batteries, which may be rechargeable, or to a rechargeable battery pack as is known by those skilled in the art depending on the implementation of the electronic reader 250.

The memory 233 is used to store the program instructions for an operating system, program code for other applications, sound files, optical measurements from previous tests, databases and a control program that the microprocessor 232 executes for controlling the operation of the electronic reader 250. The programs comprise program code that, when executed, configures the microprocessor 232 to operate in a particular manner to implement various functions for the electronic reader 250. The memory 233 may be implemented using any suitable memory device that provides suitable storage as well as volatile and nonvolatile memory elements.

Accordingly, the electronic reader embodiments described herein may be implemented, at least in part, by using one or more computer programs, executing on one or more programmable devices comprising at least one processing element and at least one storage element (i.e., at least one volatile memory element and at least one non-volatile memory element). The elements that are implemented via software may be written in a high-level procedural language such as object oriented programming. The program code may be written in MATLAB, C, C#, C++, Java, JavaScript, or any other suitable programming language and may comprise modules or classes, as is known to those skilled in object oriented programming. Alternatively, or in addition thereto, some of these elements implemented via software may be written in assembly language, machine language or firmware as needed. In either case, the language may be a compiled or interpreted language.

At least some of these software programs may be stored on a computer readable medium such as, but not limited to, a Read Only Memory (ROM) and the like that is readable by a device having a processor, an operating system, and the associated hardware and software that is necessary to implement the functionality of at least one of the embodiments described herein. The software program code, when read by the device, configures the device to operate in a new, specific, and predefined manner (e.g., as a specific purpose computer) in order to perform at least one of the methods described herein.

At least some of the programs associated with the devices, systems, and methods of the embodiments described herein may be capable of being distributed in a computer program product comprising a computer readable medium that bears computer usable instructions, such as program code, for one or more microprocessors. The medium may be provided in various forms, including non-transitory forms such as, but not limited to, one or more diskettes, compact disks, tapes, chips, cloud storage, and magnetic and electronic storage. In alternative embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

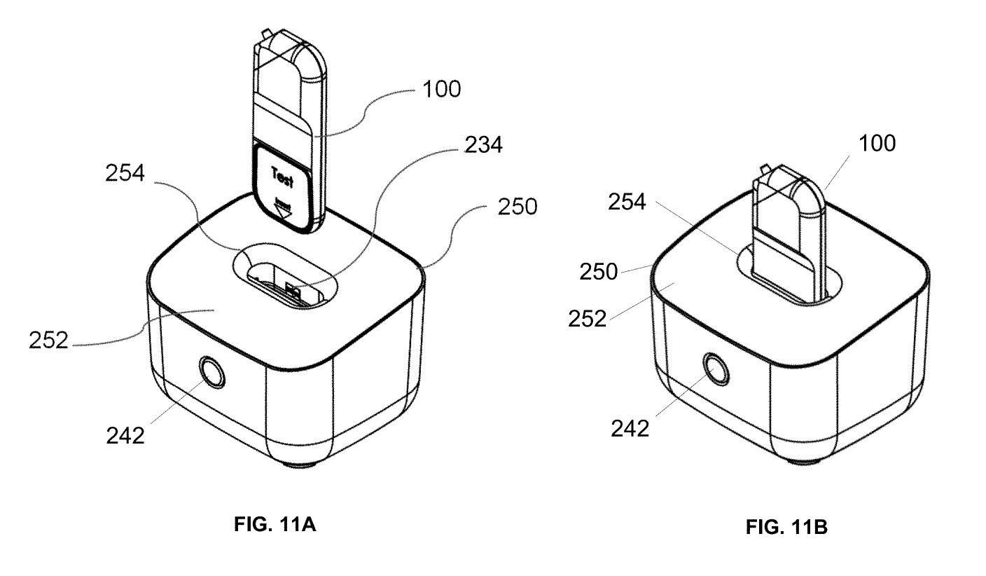

Referring now to FIGS. 11A-11B and 12A-12B, the electronic reader 250 comprises an upper surface 252 with an entrance 254 for receiving the cartridge 100 during use. After the sample 50 has been provided to the cartridge 100, the user 248 inserts the cartridge 100 into the electronic reader 250 when the user 248 is ready to begin the test. The entrance 254 of the electronic reader 250 is an aperture or slot that may be “keyed” or shaped such that the cartridge 100 can only be inserted into the electronic reader 250 in the correct orientation. The detent 144 provides tactile feedback to the user 248 that the cartridge 100 has been fully inserted into the electronic reader 250 as was previously described. A tab or boss 258 at the bottom of the entrance 254 is dimensioned such that when the cartridge 100 is inserted into the electronic reader 250, the tab 258 enters the aperture 186 of the test cartridge 100. The tab 258 then presses against the wall 152 on the strip holder piece 108 which moves the test strip 10 and the test strip holder into the post actuation position which begins the assay. The movement of the test strip 10 and the test strip holder due to the tab 258 pushing against the wall 152 also aligns the reaction zones 22 of the test strip 10 with the stripholder aperture 185 and with the optical path of the optical system 234.

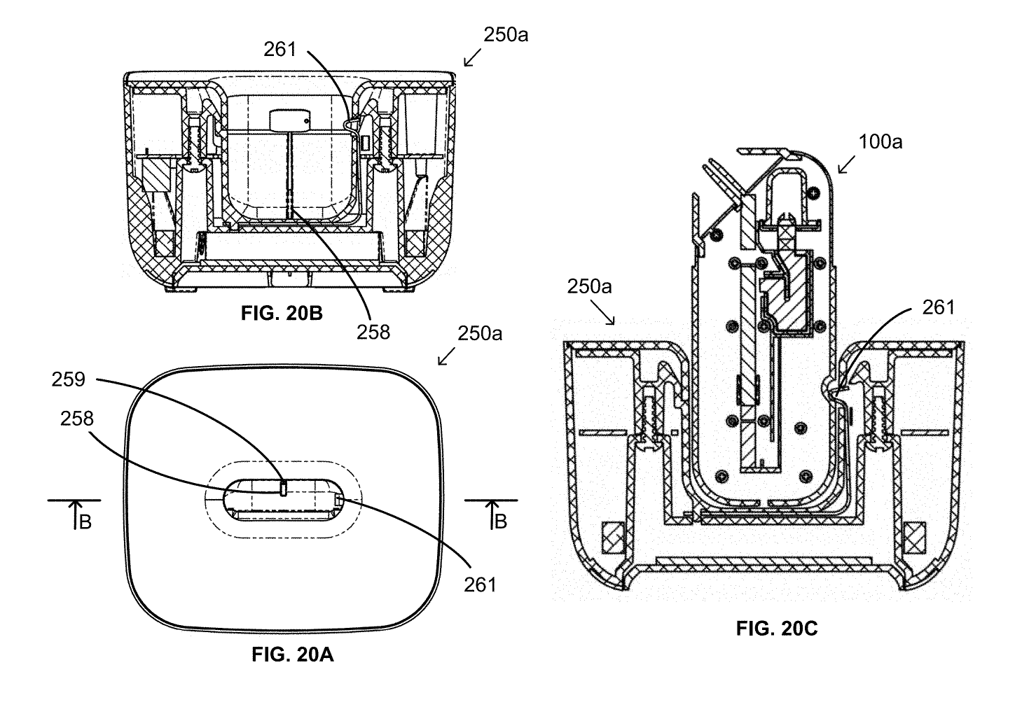

In an alternative embodiment of the electronic reader 250a, see for example FIGS. 20A-20C, the electronic reader 250a may include a rib 259. At least a portion of the rib 259 may extend between a side wall defining the entrance 254 of the electronic reader 250a and the tab 258. In some examples of the electronic reader 250a, the rib 259 may have a height/length greater than that of the tab 258. In some examples of the electronic reader 250a, the rib 259 and the tab 258 may be integrally formed. The electronic reader 250a can be used with an alternative embodiment of the cartridge, i.e. cartridge 100a.

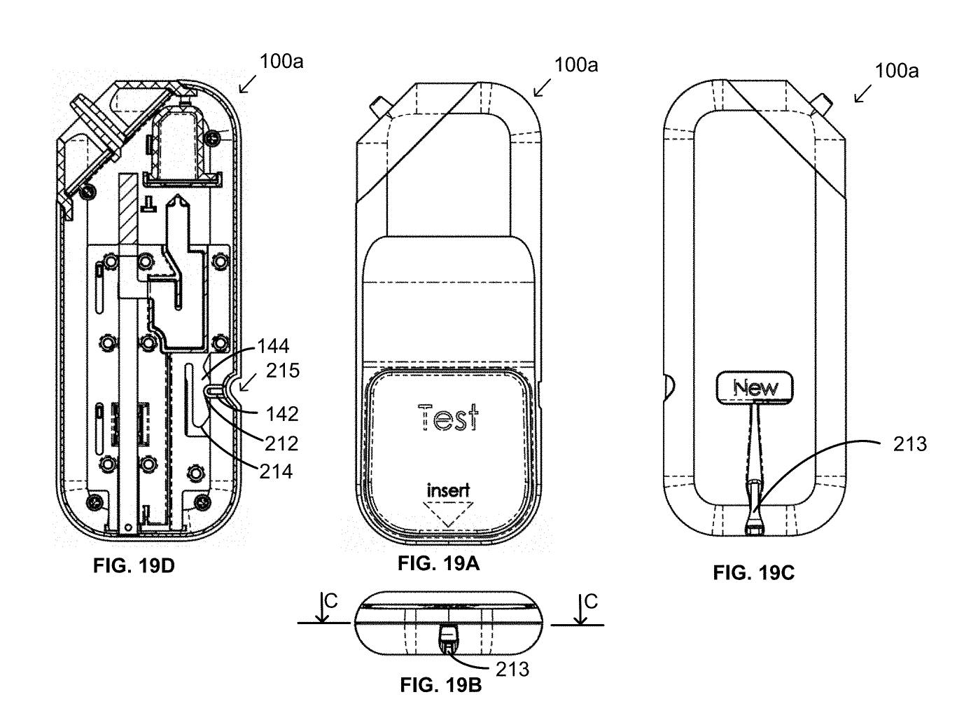

Referring now to FIGS. 19A-19D, the cartridge 100a may include a slot 213 in the casing 102 shaped to engage the rib 259. The rib 259 and the slot 213 may help align the cartridge 100a as a user inserts the cartridge 100a in the electronic reader 250a and may ensure the tab 258 aligns with the flange 152 on the strip holder. For example, when a user inserts the cartridge 100a into the electronic reader 250a, the rib 259 may first engage with the slot 213. The engagement of the rib 259 with the slot 213 may position the cartridge 100a such that when a user continues to insert the cartridge 100a into the electronic reader 250a, the tab 258 properly aligns with the flange 152.

Referring to FIG. 20C, the electronic reader 250a may also include a locator 261 that may indicate to a user that the cartridge 100a is properly inserted into the electronic reader 250. In some embodiments, see for example FIGS. 20A-20C, the locator 261 may be a leaf spring. The cartridge 100a may include a notch 215 to engage with the locator 261 as is shown in the alternative example embodiment of the cartridge 100a. The profile of the end of the locator 261 may be shaped to fit into the notch 215 of the cartridge 100a when the cartridge 100a is fully inserted.

In embodiments where the electronic reader 250a includes the rib 259 and the locator 261 is a leaf spring, and the cartridge 100a includes the slot 213, the locator 261 may apply a horizontal and downward force on the cartridge 100a, which may push the slot 213 of the cartridge 100a into the rib 259. Pressing the slot 213 of the cartridge 100a into the rib 259 of the electronic reader 250a may ensure the optical system 234 aligns with the window 185 in the cartridge 100a. The spring force provided by the locator 261 also ensures the cartridge does not move, wiggle, or fall out of the electronic reader 250a unintentionally.

Reflective photometry may be used by the electronic reader 250 to determine the quantity of label during an assay by measuring color intensity. Additionally, fluorescence measurements can be used for a fluorescent tag. Referring now to FIGS. 13A-13B, shown therein is an example embodiment of an optical system 260 that can be used by the electronic reader 250. The optical system 260 comprises a photodiode 270, an optical filter 272, at least one LED 274, intake lens 276, a lightguide system 262, an illumination lens 266, and a focusing lens 268 that are mounted on a substrate 264. The substrate 264 may be a Printed Circuit Board (PCB) or Flex Printed Circuit (FPC). In an alternative embodiment, the optical system 260 can consist of light mask acting as an optical aperture to direct light instead of or in combination with a lightguide system 262.

The optical filter 272 may be placed over or integrated into the photodetector (e.g. photodiode) 270. The photodiode 270 may be a large area photodiode and the optical filter 272 may be a red filter to enhance the blue spectrum absorption. For example, the photodiode 270 may be the PDB-C171SM from Luna Optoelectronics. The photodiode 270 and the optical filter 272 are spaced apart from the focusing lens 268 in the axial direction. The central axes of the photodiode 270, the optical filter 272 and the focusing lens 268 are aligned with one another. During use, the photodiode 270 generates a current that is proportional to the reflected light that it senses. The generated current is then amplified and converted to a digital voltage reading which can be read and interpreted by the microprocessor 232.

In alternative embodiments, other photodiodes may be used with sensitivity to other regions of the light spectrum and depends on the type of test being performed by the cartridge 100. In different embodiments, a camera, a photodiode or a CCD may be used to capture the light that reflects from the test strip 10 during measurement of the test results. In different embodiments, a second photodiode or multiple photodiodes can be used to capture light directly from the LEDs providing a reference channel for the system.

In this example embodiment, the optical system 260 comprises two LEDs 274 mounted on the PCB 264. In FIG. 13B, only one of the LEDs 274 is visible since they are collinear with one another. Alternatively, in some embodiments, multiple (e.g. more than two) LEDs may be used to provide a wide breadth of illumination. In some embodiments, there may be one LED that is used for each reaction zone 22 on the reaction membrane 21.

The LEDs 274 are laterally spaced apart from the photodiode 270 and optical filter 272. The LEDs 274 are also spaced apart from the intake lens 276. The intake lens 276 has a certain orientation and is optically coupled to the lightguide system 262 and the illumination lens 266 in order to focus and transmit the generated light to the reaction zones 22 of the test strip 10. Preferably, the LEDs 274 may be selected to have a sufficient level of brightness, in order to increase signal to background light, and a narrow emission angle to ensure that most of the light enters into and reflects internally within the lightguide system 262.

The LEDs 274 are also selected such that the wavelengths of the light generated by the LED 274 match the absorbance spectrum of the tagging agent, or color change, observed on the strip 10 due to the assay that is performed. For example, a bright green LED, such as the QBLP653-IG LED from QT Brightek, may be used since it has a discrete emission with a peak intensity at 525 nm, which matches the absorption of gold nanoparticles, used as a tagging agent for some tests. For other tagging agents a different LED and photodiode combination may be used such that the LED emission peak overlaps the absorbance spectrum of the tagging agent and the photodiode 270 has high sensitivity in that same region.

The lightguide system 262 of the optical assembly 260 comprises two light paths LP1 and LP2, one for each LED 274, so that each LED 274 illuminates one of the two reaction zones 22 on the test strip 10. In other embodiments, there may be additional light paths and LEDs to allow the measurement of more than two reaction zones on the test strip. The lightguide system 262 that provides the light paths LP1 and LP2 comprises two optical conduits that are parallel to one another and aligned with the reaction zones 22 that are to be illuminated on the test strip. The optical conduits are made from a transparent polymer with a high refractive index such as polycarbonate to maximize internal reflection. The light paths LP1 and LP2 provided by the two optical conduits are labelled with dashed lines on FIGS. 13A and 13B. A piece of black plastic, shown by the shaded regions in FIG. 13B, shields the two optical paths from each other and reduces unwanted ambient light. The reflected light that occurs when the reaction zones 22 are illuminated goes to the same photodetector 270 and therefore only one of the LEDs 274 is turned on at any one time. This configuration is advantageous since an additional photodiode and supporting amplifier circuit are not used, which reduces cost. Furthermore, with this configuration any biasing or drift of the photodiode current will be reflected equally in the measurements regardless of which LED 274 provides the illumination when measuring the test results. When one of the LEDs 274 illuminates the reaction zone 22 that acts as a control, the reflected and measured light signal is the control signal. When the other of the LEDs 274 illuminates the reaction zone 22 that is used to measure the analyte of interest in the sample 50, the reflected light signal is the analyte signal.

FIGS. 8C-8F, FIGS. 8C and 8D show longitudinal and transverse cross-sectional views, respectively, of the cartridge 100 while FIGS. 8E and 8F show enlarged views of portions of FIGS. 8C and 8D, respectively. During an assay, a light stimulus L1 that is emitted from one of the LEDs 274 shines through the optical opening 185 in the cartridge 100. The light emitted from the LED 274 is directed towards one of the reaction zones 22 (referred to as a first reaction zone) on the reaction membrane 21 of the test strip 10 by the lightguide 262 system. The light L1 enters the lightguide system 262 through the intake lens 276 and is reflected internally within the lightguide system 262 exiting at the illumination lens 268. The illumination lens 268 focuses the light stimulus L1 from the LED 274 to the first reaction zone of the test strip 10. Portions of the strip holder 108 surround the first reaction zone of the test strip 10 in order to prevent the reflection of light from other areas of the cartridge 100 or the test strip 10 in order to perform a valid test. The light L1 enters the first reaction zone of the test strip 10, is absorbed, and is scattered as light R1. The scattered light R1 travels out of the opening 185 through the focusing lens 268 on the lightguide 262 and towards the optical filter 272 and the photodetector 270. The process is repeated using the second LED 274 to generate the second light signal L2 that is directed to the other of the reaction zones 22 (referred to as a second reaction zone) to generate the second scattered light signal R2. As described previously, one of the light signals R1 and R2 is the control signal and the other is the analyte signal (also known as the test signal) depending on which of the first and second target reaction zones act as a control. Also, as discussed previously, the light signals L1 and L2 are generated and provided to the reaction zones 22 one at a time. FIGS. 13A-13B show R1 and R2.

The electronic reader 250 is able to monitor the progress of the assay in the cartridge 100 by monitoring data from the optical system 234. The electronic reader 250 can use the display 244 to communicate to the user 248 when a test has started, what the test progress is and if any errors occur. Other methods of notification include sound (through the speaker 245), vibration or wireless communication (through the Bluetooth module 246) to a separate device such as the phone 247 or a computing device like a server, laptop or tablet, for example. The microprocessor 232 is sensing data values from the optical system 234 periodically, such as every 10 ms, 100 ms, 1 second, or 5 seconds, for example. The microprocessor 232 can determine the rate of change and magnitude of these data value and compare them to the amount of time that the test has been operating for to look for unacceptable variabilities. Accordingly, the microprocessor 232 of the electronic reader 250 can compare the progression of the optical measurements with what is expected. For example, the electronic reader 250 may first determine a baseline reading when the cartridge 100 is inserted into the opening 254. If the baseline reading is out of a specified range it can be assumed that the cartridge 100 is faulty or is inserted improperly and the electronic reader 250 will notify the user 248 about this situation. In the same manner, the electronic reader 250 can confirm that the sample 50 has entered the test strip 10 and if the buffer liquid 162 has released properly by comparing the optical results, provided by the optical system 234 during the test, with expected values at the corresponding time of the test. If there are sudden and unexpected changes in the development of the test signal and/or the control signal, the electronic reader 250 can notify the user of the error.

In this example embodiment, the electronic reader 250 can be connected to a smart device, such as the phone 247, via the Bluetooth module 246 to send the test results to the smart device. The test results can be saved on the smart device 247 or another electronic device such as, but not limited to, a hospital electronic record system, a tablet, a laptop, or a remote server on the cloud, for example.

In this example embodiment, the NFC tag 126 that is attached to the cartridge 100 can be read by the NFC reader 240. The NFC reader 240 includes an NFC reader antenna and a circuit to be able to read data from the NFC tag 126. The NFC reader antenna is fixed in the electronic reader 250 such that it is positioned parallel to the NFC tag 126 on the cartridge 100. The NFC reader 240 monitors the NFC reader antenna for voltage changes in a received signal while the electronic reader 250 is turned on. This serves a few purposes. The electronic reader 250 can detect the presence of the NFC tag 126 and determine when the cartridge 100 is inserted by the proximity of the NFC tag 126 to the NFC reader antenna. The electronic reader 250, knowing that the cartridge 100 has been inserted, can then turn on from a sleeping state to a test state. During the test state, the electronic reader 250 turns on the LEDs 236 and begins monitoring the optical system 234 (i.e. the photodetector 270) for optical signals conveying the status of the test and the test results as described in the previous paragraph.

The NFC tag 126 can also store information about the cartridge 100, which is unique to the cartridge 100, such as the type of test that can be performed by the cartridge 100. Storing the type of test allows the electronic reader 250 to accept cartridges for testing for different biomarkers in a sample. A manufacturing date and test ID for the cartridge 100 can be used to determine whether the electronic reader 250 is compatible with a particular cartridge 100. An expiry date can prevent an expired cartridge from being used with the electronic reader 250 which prevents inaccurate results.

In some embodiments, the information that can be used to interpret the test results from the optical measurements made on the cartridge 100 can be stored in the electronic reader 250. This information can include an equation that is used to interpret the optical signal formation at the reaction zones 22 and correlate it to a concentration value. This equation may be different from cartridge to cartridge that perform the same test due to variations in manufacturing. Accordingly, during manufacturing, “standards” or samples containing known concentrations of a marker used in the test will be measured using the electronic reader 250 to determine the parameters for this equation. Small changes in the manufacturing process means that this equation can change slightly so it may be determined and stored at the production of each batch of cartridges. Enough information is included on the NFC tag 126, including the parameters used in the equation to correlate optical measurements to biomarker concentration so that the electronic reader 250 can use unique batch information on the NFC tag 126 to determine the test results. In some embodiments, the name of the test, the biomarker and the measurements units can be stored on the NFC tag 126, read by the microprocessor 232 and then shown on the display 244. This allows future test cartridges 100 that are produced to be compatible with an older electronic reader 250 without having to perform an additional update on the electronic reader 250.

Similarly, in an alternative embodiment, the identification of the cartridge 100 and the test information can be encoded on a QR code that is on the outside of the cartridge. During testing and measurements, when the cartridge having the QR code is inserted into the electronic reader, the QR code can be read by a camera or a laser scanner that is included in the electronic reader 250. In an alternative embodiment, a memory chip can be embedded into the cartridge 100 to communicate with the electronic reader 250 by contact after or during insertion of the cartridge 100 into the electronic reader 250. In this case, the electronic reader 250 may include metal spring contacts that make contact with metal contacts on the cartridge 100 in order to transfer electronic data that is stored in the cartridge to the electronic reader. In another alternative embodiment, the QR code may be printed on the test cartridge or a package for the test cartridge and read by a smartphone application or the electronic reader 250. In this alternative embodiment, the QR code reader is located on the outside surface of the electronic reader 250. In another alternative embodiment, the cartridge and test information may be stored on a separate chip that is packaged with the cartridge and is inserted into the electronic reader 250 when testing is performed on the cartridge 100.

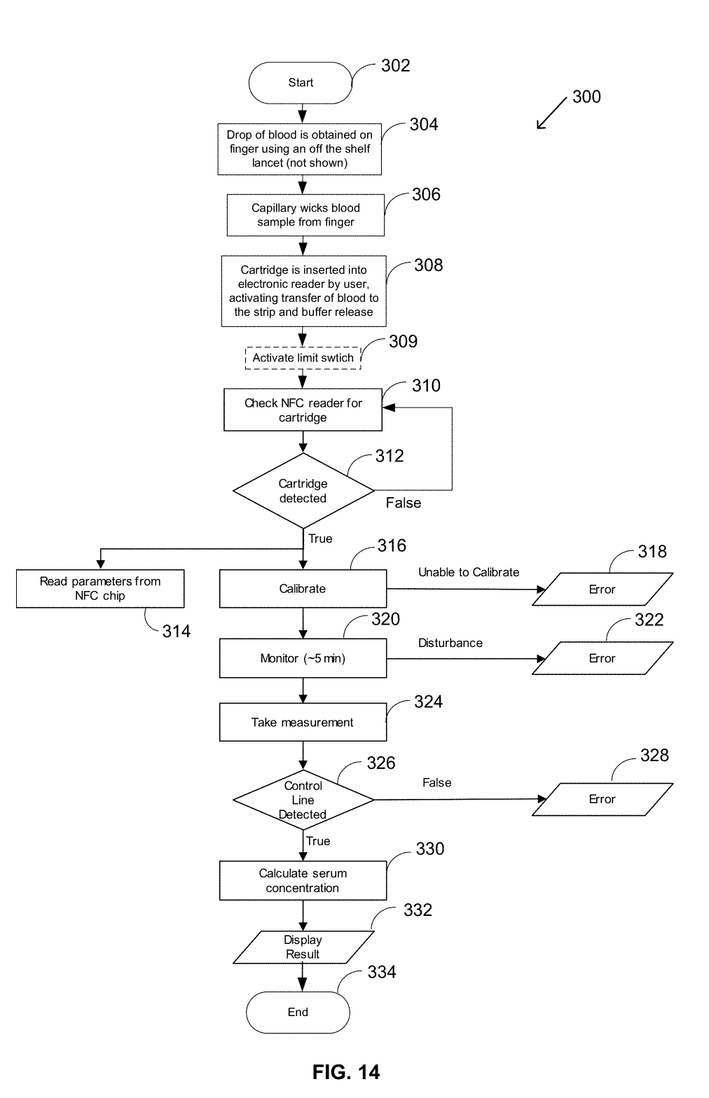

Referring now to FIG. 14, shown therein is a flow chart of an example embodiment of a method 300 of performing an assay using a cartridge and an electronic reader in accordance with the teachings herein.

At act 304, the sample 50 that will be tested is obtained. For example, if the sample 50 is blood then a drop of blood can be obtained from a patient's figure using a sharp object such as an off the shelf lancet, for example.

At act 306, the sample 50 is provided to the cartridge 100. For example, as shown in FIG. 15, the patient's finger with a drop of blood on it can be brought in close proximity to the capillary assembly 106 of the cartridge 100 so that the capillary tube 182 can wick-up the blood sample. This continues until a sufficient amount of the sample 50 is within the capillary tube 182.

When the user 248 is ready to start using the electronic reader 250, they press the on/off button 242 for a certain amount of time, such as 2 seconds, for example. When the electronic reader 250 turns on and is ready to start a test, it may indicate this to the user 248. For example, the electronic reader 250 may show a message on the display 244 such as “Insert Cartridge” or “Begin test”. In some embodiments, the electronic reader 250 may perform some calibration or diagnostic tests after turning on and before indicating that it is ready to perform the test. For example, in some instances, the electronic reader 250 may be able to detect current ambient conditions, such as ambient light or ambient temperature.

At act 308, the user 248 inserts the cartridge 100 into the entrance of the slot 254 of the electronic reader 250. The tab 234 at the bottom of the slot 254 actuates the strip holder of the cartridge 100. The strip holder then moves from the pre-actuation position to the post-actuation position thereby: (1) activating the transfer of the blood sample 50 from the capillary tube 182 to the test strip 10 and (2) puncturing the buffer capsule 116 to release the buffer liquid 162 to the buffer membrane 114.

As described above, in some embodiments the electronic reader 250 may include a locator (see locator 261 in FIGS. 20A-20C). The locator may include a leaf spring that is contoured to match an indent in the cartridge. The locator may be connected to a limit switch which may detect the position of the locator. In such an embodiment, there may be an additional act 309 of activating the limit switch. In embodiments in which the limit switch is not included the act 309 is not included. Accordingly, act 309 may be optional in some cases depending on the embodiment of the electronic reader 250. The limit switch may be activated by a cartridge 100 being inserted into the electronic reader 250. When activated, the limit switch may signal the electronic reader 250 that a cartridge 100 has been inserted into the electronic reader 250. When signaled by the limit switch that a cartridge 100 has been inserted, the electronic reader 250 will proceed to act 310 described below. If the electronic reader 250 does not include a limit switch, the electronic reader 250 may continuously scan for a NFC tag as described below.

At act 310, the electronic reader 250 detects whether the cartridge 100 that was inserted into the electronic reader 250 has the NFC tag 126. If the cartridge 100 has the NFC tag 126, then the electronic reader 250 detects the cartridge 100 at act 312. The method 300 then proceeds to act 314 where the electronic reader 250 reads data from the NFC tag 126 including the type of test to be performed and parameters that can be used to determine the assay results from the measurements.

In an alternative embodiment where the electronic reader 250 cannot detect and read the NFC tag 126 or the cartridge 100 does not have the NFC tag 126, acts 310 to 314 of the method 300 do not exist. Rather, the electronic reader 250 is modified to already store the parameters needed to perform a variety of tests in internal memory. The method 300 then involves reading the data needed to perform the test based on information about the test (i.e. test type) that can be inputted by the user 248 via an input interface (such as the display 244 when it is a touch display).

In both of these cases, the electronic reader 250 may then show on the display 244 the name of the test that is being performed. For example, if the sample 50 is being analyzed for its concentration of Vitamin D, the electronic reader 250 may show the text “Vitamin D” on the display 244.

At act 316, the electronic reader 250 calibrates the optical system 234 to obtain a baseline for the cartridge 100 before any liquid enters the reaction membrane 21 of the test strip 10. If the baseline reading is not within an acceptable range this can indicate an issue with the test strip 10 and the method 300 proceeds to act 318 where an error for the assay is indicated. This may be indicated using the display 244 or the speaker 245 of the electronic reader 250 or it can be delivered in an electronic message that is sent by the Bluetooth module 246.

At act 320, the electronic reader 250 acquires one or more test signals (one for each LED used to measure an analyte of interest) using the optical system 234 and monitors these signals to confirm that the test progresses as intended. The electronic reader 250 can see an abrupt drop in reflectivity when liquid passes through the test strip 10 that is visible at the optical window 185. The timing of this indicates whether the sample 50 has successfully entered the reaction membrane 21 of the test strip 10. If this drop in reflectivity is not detected, then the method 300 may proceed to act 322 where a corresponding error is reported.

Still at act 324, the electronic reader 250 waits for the test signal(s) to stabilize indicating that the reaction is complete and the reagents have been washed out of the reaction membrane 21. For example, the acquired test signal(s) may slowly develop over a period of time, such as 5 minutes for example, and then converge on a result (i.e. a steady-state amplitude where there is less than about +/−2% signal amplitude variation, +/−1% signal amplitude variation or +/−0.5% signal amplitude variation). If this does not happen in a timely fashion then the method 300 may proceed to act 322 where a corresponding error is reported.

In an alternative embodiment, the electronic reader 250 may employ a timer or the microprocessor 232 may run a clock program to count down from a predetermined test duration to zero in order to determine when it is ready to begin measuring the test signals from the cartridge 100.

In another alternative embodiment, the electronic reader 250 may use the countdown technique as well as monitor the optical signals to determine when it can take measurements on the test signal in order to determine the test result. In this case, if the timer counts down to zero before the optical signals have reached a steady-state level then this may indicate that there is an error in the test.

Before the test is completed, it may be possible that the user 248 attempts to remove or accidentally agitates the test cartridge 100 from the electronic reader 250. For example, the electronic reader 250 may include a motion sensor that can detect movement of the test cartridge 100 during testing. Alternatively, or in addition thereto, the NFC reader 240 may be used to detect motion of the test cartridge 100. In either of these cases, the electronic reader 250 may warn the user 248 by showing appropriate text or an image on the display 244 and/or making a sound using the speaker 245. At this point, the user 248 may remove the test cartridge 100 to reduce the risk that the test is performed incorrectly. In this case, the method 300 ends. Alternatively, if the test cartridge 100 is not removed then the method 300 continues.

Once the test signal has stabilized, the method 300 proceeds to act 324 where measurements are made on the test signal, which are then used to determine the test results. For example, a moving average may be used on the acquired test signal(s) to smooth out the measured optical data. In addition, to avoid the influence of changes in ambient light measurements can be made when the LED is periodically turned off and on to measure the influence of changes in ambient light from the surrounding environment. The test signal may be measured before the control signal, because a certain period of time elapses, such as 5 minutes for example, before measurement of the test signal in order to establish its steady-state.

At act 326, the method 300 determines whether the control signal has been detected via the optical system 234 and if not indicates a corresponding error at 328. In this error, if the photo-detector 238 does not measure any development of the control signal in the reaction zone 22 that is used to provide a control (i.e. the “control reaction zone”), the error will read that the test cartridge 100 is faulty. If a control signal should have developed in the control reaction zone but did not form, as determined by the tagging agent entering the control reaction zone, which can be confirmed optically, then the error will read that there was insufficient sample 50 placed in the capillary tube 182.

If the control signal is detected at act 326, the method 300 proceeds to act 330, where the electronic reader 250 where the analyte measurements obtained at act 324 are adjusted for any measurement biases. For example, the calibration baseline measurement and any change in ambient light may be subtracted from the analyte measurements to obtain an adjusted test reading. The ambient light may be measured by taking optical measurements when all of the LEDs 274 are off. The electronic reader 250 then uses the adjusted test reading to determine a serum concentration (which is known as the assay result) using the test parameters (which may be defined by the NFC tag 126.

In some embodiments, after the test is completed, the electronic reader 250 may show the message “Complete” on the display 244. In some embodiments, the electronic reader 250 may instead, or additionally, make a sound using the speaker 245 to notify the user 248 that the test is complete.

At act 332, after the test is complete the assay result is provided to the user 248. This may be done by showing the assay result on the display 244. Alternatively, or in addition thereto, the electronic reader 250 can transmit the assay result to the smartphone 247, a computer or another electronic device via Bluetooth. Alternatively, other communication methods may be used such as WiFi, cellular connection or a physical cable, as is known by those skilled in the art.

At act 334, the test is ended and a number of further actions can be performed. For example, the electronic reader 250 may prompt the user 248 to remove the test cartridge 100 by showing the message “Test Complete” and/or “Remove Cartridge” on the display 244.

Once the user 248 has removed the test cartridge 100, the electronic reader 250 may then show the message “Ready for new test” on the display 244. In some embodiments, this text may be alternated with displaying the last test result. In some embodiments, before the electronic reader 250 indicates that it is ready to perform another test, it may perform some internal diagnostics to make sure that it is able to perform another test. For example, the electronic reader 250 may check the battery level and/or internal temperature to see if these parameters are in a normal operational range.

In some embodiments, after the test cartridge 100 is removed and there is a certain amount of inactivity, such as 5 minutes for example, the electronic reader 250 may start blinking some text or images on the display 244. If the user 248 wishes to turn off the electronic reader 250, then the user 248 may hold down the on/off button 242 for a certain amount of time. The electronic reader 250 then powers down completely at that time.

Alternatively, if the user 248 does not take any action and the test cartridge 100 is left in the electronic reader 250 for a certain period of time, such as 5 minutes for example, then the electronic reader 250 may enter a sleep mode until the test cartridge 100 is removed. During sleep mode, the electronic reader 250 uses minimal power but the electronic reader 250 monitors the test cartridge 100 to determine if it is still inserted. For example, the photodetector 238 may be used to continue to monitor ambient light and when the test cartridge 100 is removed, there will be a higher ambient light that will be detected by the photodetector 238.

Alternatively, in some embodiments, when the electronic reader 250 is in sleep mode, the user 248 may press the on/off button 242 for a short amount of time (e.g. a short button press less than 2 seconds). The electronic reader 250 then powers up and may display the last test results.

Alternatively, in some embodiments, when the electronic reader 250 is in sleep mode, the user 248 may press the on/off button 242 for a longer amount of time (e.g. a long button press longer than 2 seconds). The electronic reader 250 then powers up and can prompt the user 248 to remove the test cartridge 100. In this case, the electronic reader 250 may also display the last test results.

It should be noted that in some embodiments, the user 248 may have paired their smart phone or another electronic device with the electronic reader 250 before the test was performed. In embodiments in which the countdown timer is used and a device is paired with the electronic reader 250, an analyte test application on the paired device may display the countdown until the test is completed. When the electronic reader 250 completes the test, it may also notify the user 248 by sending a message to the paired device. The message is read by the analyte test application and a corresponding text message or image is displayed on the paired device to indicate that the test is complete. In some embodiments in which there is a paired device and analyte test application, the electronic reader 250 may also send the test results to the paired device which may then be stored and/or displayed on the paired device by the analyte test application.

In an alternative embodiment, the user 248 may insert the test cartridge 100 into the electronic reader 250 while the electronic reader 250 is off. The electronic reader 250 is able to detect that the test cartridge 100 has been inserted and then turns on immediately. The electronic reader 250 may determine this by monitoring for changes in ambient light (which will decrease substantially after the test cartridge 100 is inserted). In some cases, the electronic reader 250 has enough time to recognize the type of test cartridge 100 that is inserted and perform calibration before the test begins. In this case, the electronic reader 250 performs the method 300 starting at act 310. However, there may be some cases in which the electronic reader 250 does not have enough time to perform calibration in which case the electronic reader 250 proceeds to act 318 of the method 300 and an error is shown on the display 244 to indicate that there is a test failure due to improper cartridge insertion, an expired test cartridge or an already used test cartridge.

It should be understood that in some embodiments there are some other situations which may occur that may compromise the integrity of performing the test using the test cartridge 100. The situations may include a low battery level, the test cartridge 100 is not operating properly, or the test cartridge 100 is expired or already used.

For example, in some embodiments, when the electronic reader 250 is turned on, it may detect that there is a low battery level when one or more batteries is used to power the electronic reader 250. The electronic reader 250 notifies user 248 that the battery level is too low to perform a test and that the one or more batteries need to be changed.

In another example, in some embodiments, the electronic reader 250 may detect that the test cartridge 100 is not working properly. For example, this may be determined by monitoring the magnitude and/or time characteristics of the test and control signals to determine if they are in normal operational ranges. The electronic reader 250 may notify the user 248 that the test has failed by showing an appropriate text message or image on the display 244. The user 248 may then remove the test cartridge 100 and discard it.

In another example, in some embodiments, the test cartridge 100 is expired or is already used and the user 248 inserts the test cartridge 100 by mistake in the electronic reader 250. For example, this may be determined by programming the expiry date onto the NFC tag 126, and the electronic reader 250 can read this data using the NFC reader 240 and compare it to current data to determine if the test cartridge has expired. In this case, the electronic reader 250 recognizes that the test cartridge 100 is expired/used and notifies the user 248 by showing a text message on the display 244 and/or making a sound using the speaker 245. The user 248 then removes and discards the test cartridge 100.

The various test strips, cartridges and electronic readers of the present disclosure can be applied to other types of samples other than blood. For example, the sample can be water, a beverage, urine, saliva, blood, semen or other biological solutions. Biomarkers (also known as analytes of interest) that can be tested for include, but are not limited to, vitamin D, 25 Hydroxy vitamin D, 1,25 Dihydroxy Vitamin D, folic acid, transferrin, iron, glucose, Tumor Growth Factor, Prostate Specific Antigen, Placenta Growth Factor,

Thyroglobulin, cholesterol, HDL cholesterol, LDL cholesterol, triglycerides, creatinine, Tetrahydrocannabinol, Cannabidiol, B vitamins, cortisol, testosterone, ferritin, and hemoglobin. The reagents deposited at the reaction zones 22 and the conjugate pad 16 can be switched out with the corresponding binding system that is appropriate for the particular biomarker that is being detected for a particular test cartridge. For instance, an antibody pair may be used to detect the ferritin protein in a sandwich immunoassay format. Alternatively, for vitamin D a single antibody may be used along with a competitive vitamin D molecule attached to BSA (Bovine Serum Albumin).

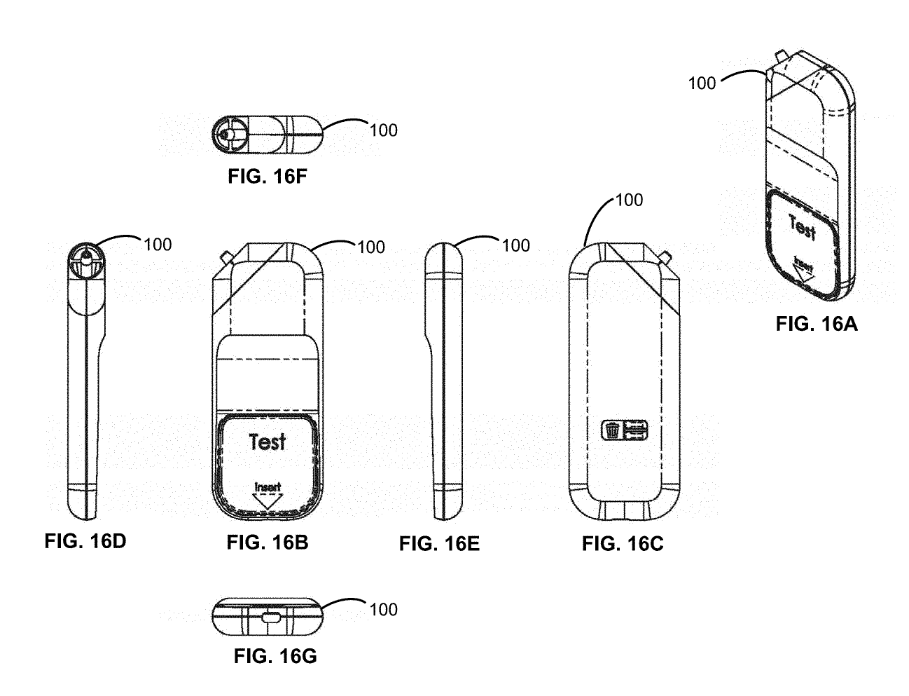

Referring now to FIGS. 16A-16G, shown therein are alternative views for illustrative purposes of the cartridge 100 for performing an assay in accordance with the teachings herein. In the drawings of the of the cartridge 100: FIG. 16A is a perspective view of the cartridge 100; FIG. 16B is a front view of the cartridge 100 of FIG. 16A; FIG. 16C is a rear view of the cartridge 100 of FIG. 16A; FIG. 16D is a left side view of the cartridge 100 of FIG. 16A; FIG. 16E is a right side view of the cartridge 100 of FIG. 16A; FIG. 16F is a bottom view of the cartridge 100 of FIG. 16A; and, FIG. 16G is a top view of the cartridge 100 of FIG. 16A.

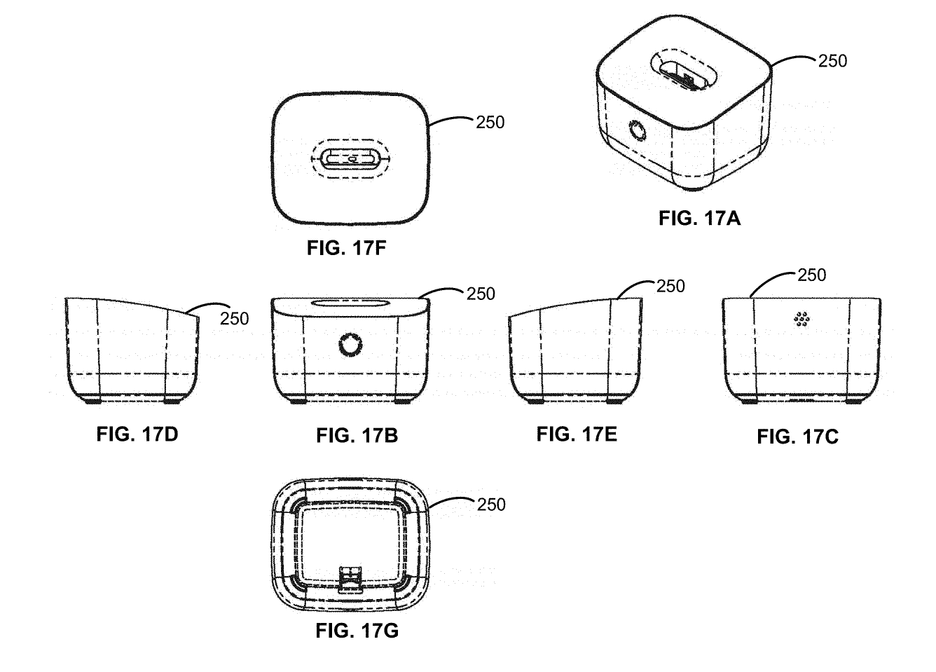

Referring now to FIGS. 17A-17G, shown therein are alternative views for illustrative purposes of the electronic reader 250 for initiating and measuring the results of an assay in accordance with the teachings herein. In the drawings of the electronic reader: FIG. 17A is a perspective view of an electronic reader 250; FIG. 17B is a front view of the electronic reader 250 of FIG. 17A; FIG. 17C is a rear view of the electronic reader 250 of FIG. 17A; FIG. 17D is a left side view of the electronic reader 250 of FIG. 17A; FIG. 17E is a right side view of the electronic reader 250 of FIG. 17A; FIG. 17F is a top view of the electronic reader 250 of FIG. 17A; and, FIG. 17G is a bottom view of the electronic reader 250 of FIG. 17A.

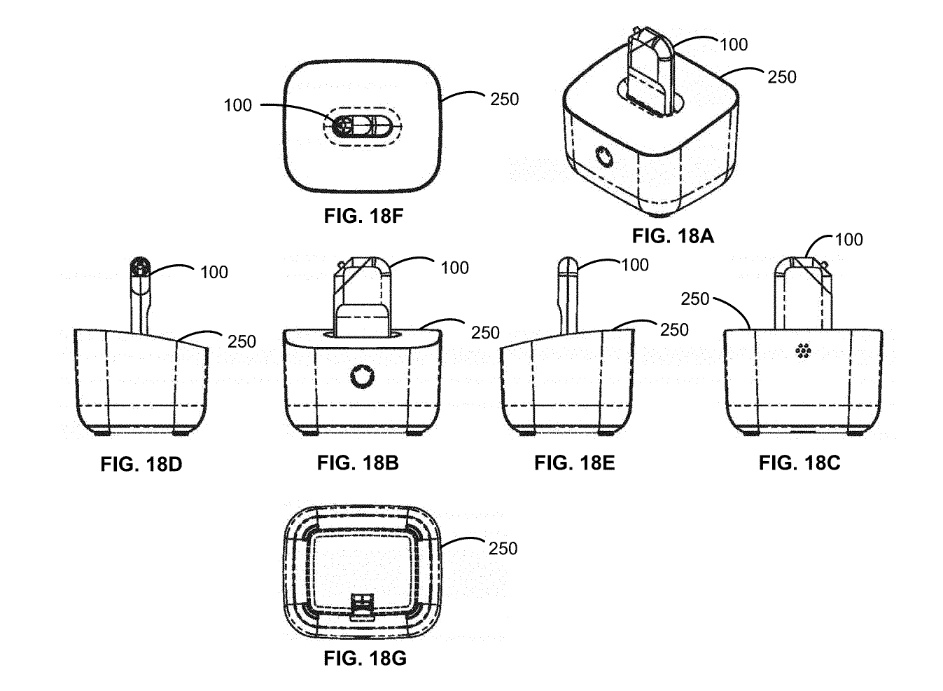

Referring now to FIGS. 18A-18G, shown therein are alternative views for illustrative purposes of the cartridge 100 and the electronic reader 250 that can be used together to perform an assay and measure the assay results in accordance with the teachings herein. In the drawings of the cartridge 100 and the electronic reader 250: FIG. 18A is a perspective view of the cartridge and electronic reader 250; FIG. 18B is a front view of the cartridge 100 and the electronic reader 250 of FIG. 18A; FIG. 18C is a rear view of the cartridge 100 and the electronic reader 250 of FIG. 18A; FIG. 18D is a left side view of the cartridge 100 and the electronic reader 250 of FIG. 18A; FIG. 18E is a right side view of the cartridge 100 and the electronic reader 250 of FIG. 18A; FIG. 18F is a top view of the cartridge 100 and the electronic reader 250 of FIG. 18A; and, FIG. 18G is a bottom view of the cartridge 100 and the electronic reader 250 of FIG. 18A.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

We claim:

1. A test cartridge that allows for an automated assay of various analytes in a sample, wherein the test cartridge comprises:
a capillary assembly to receive, store and release the sample;
a buffer capsule for storing and releasing a buffer liquid;
a buffer membrane to receive the buffer liquid during the assay;
a test strip that is coupled to the buffer membrane and adapted to perform the assay after receiving the sample and the buffer liquid;
a test strip holder adapted to hold the test strip and the buffer membrane and move towards the capillary assembly and the buffer membrane to provide the sample to the test strip and the buffer liquid to the buffer membrane when the assay is performed; and
an enclosure for providing a housing for the test cartridge.

2. The test cartridge of claim 1, wherein the test strip holder is adapted to move from a pre-actuation position to a post-actuation position, wherein in the pre-actuation position the test strip and the buffer membrane are spaced apart from the capillary assembly and the buffer capsule, respectively, and in the post-actuation position the test strip is brought into contact with the capillary assembly to receive the sample, the buffer membrane is brought into contact with the buffer capsule to receive the buffer liquid which is then transferred to the test strip and the test strip performs the assay.

3. The test cartridge of claim 1, further comprising a sliding rail system made of slots formed in one surface of the test strip holder and pegs formed in an opposing surface of the enclosure and the test strip holder is adapted to move along the sliding rail system.

4. The test cartridge of claim 3, wherein the enclosure comprises an aperture opposite a bottom end of the test strip holder for receiving a force to move the test strip holder from the pre-actuation position to the post-actuation position.

5. The test cartridge of claim 1, wherein the capillary assembly is located at an upper corner of the enclosure and comprises:
an outer facing end having a first aperture open to an exterior of the enclosure;
an inner facing end having a second aperture open to an interior of the enclosure; and
a capillary tube between the outer facing end and the inner facing end, the capillary tube drawing the sample into the capillary assembly using capillary forces during use.

6. The test cartridge of claim 1, wherein the test strip comprises:
a backing card for providing support for membranes of the test strip;
a reaction membrane disposed on the backing card, the reaction membrane having reaction zones including a control zone for providing control measurements and at least one assay reaction zone for reacting with at least one analyte of interest;
a conjugate pad overlaid on a distal portion of the reaction membrane, the conjugate pad containing chemicals for performing the assay; and
a sample pad overlaid on a distal portion of the conjugate pad and wrapped around a back surface of the backing card and having a rounded end at a distal portion of the test strip, the sample pad having a filter membrane disposed adjacent the rounded end for receiving the sample and having a buffer entrance area disposed near the back surface of the backing card for receiving the buffer liquid.

7. The test cartridge of claim 6, wherein the chemicals of the conjugate pad comprise at least one of buffers, surfactants, salts, antibodies and labelling agents.

8. The test cartridge of claim 6, wherein when the test strip holder is moved to the post-actuation position, the rounded end of the sample pad is moved to the inner facing end of the capillary assembly to receive the sample from the capillary tube.

9. The test cartridge of claim 6, wherein the buffer membrane comprises:
a buffer intake area to receive the buffer liquid from the buffer capsule;
a U-shaped bottom region downstream of the buffer intake area where the buffer liquid pools as the buffer liquid drawn from the buffer capsule;
a junction region downstream of the bottom region and adjacent to the test strip; and
an exit region comprising an arm adjacent to and downstream of the junction region, the exit region overlapping with the buffer entrance area of the test strip to provide the buffer liquid thereto.

10. The test cartridge of claim 9, wherein the test strip holder comprises a protrusion that is moved into an end of the buffer capsule to release the buffer liquid to the buffer membrane when the test strip holder is moved to the post-actuation position.

11. The test cartridge of claim 9, wherein the buffer membrane comprises an arm portion that is fluidly coupled to the test strip to provide the buffer liquid thereto, the arm portion having a size, shape and material that are selected to provide a desired flow rate to delay the arrival of the buffer liquid into the test strip.

12. The test cartridge of claim 6, wherein the enclosure comprises an optical window that is aligned with the reaction zones of the reaction membrane when the test strip holder is moved to the post-actuation position to allow for optical measurements of the reaction zones during assay results measurement.

13. The test cartridge of claim 1, wherein the enclosure comprises a first indicator to indicate when the test cartridge is new and when it is used.

14. The test cartridge of claim 1, wherein the enclosure comprises a second indicator to indicate when the test cartridge is expired.

15. The test cartridge of claim 1, wherein the enclosure comprises a third indicator to indicate when the test cartridge is exposed to prohibited humidity or temperature during storage.

16. The test cartridge of claim 1, wherein the enclosure comprises first and second enclosure pieces to provide the housing for the test cartridge to protect the test strip from contamination or exposure to environmental humidity.

17. The test cartridge of claim 1, wherein the test cartridge further comprises an NFC tag disposed with the enclosure, the NFC tag being adapted to store information about the test cartridge including information about at least one of a type of assay that is performed by the test cartridge and parameters that are used to determine the assay results from assay measurements.

18. The test cartridge of claim 1, wherein the test strip and the buffer membrane are disposed within the test cartridge to perform a lateral flow assay.

19. An electronic reader for performing an assay test and measuring test results, wherein the electronic reader comprises:
a slot for receiving a test cartridge that allows for an automated assay of various analytes in a sample, wherein the test cartridge comprises:
a capillary assembly to receive, store and release the sample;
a buffer capsule for storing and releasing a buffer liquid;
a buffer membrane to receive the buffer liquid during the assay;
a test strip that is coupled to the buffer membrane and adapted to perform the assay after receiving the sample and the buffer liquid;
a test strip holder adapted to hold the test strip and the buffer membrane and move towards the capillary assembly and the buffer membrane to provide the sample to the test strip and the buffer liquid to the buffer membrane when the assay is performed; and
an enclosure for providing a housing for the test cartridge;
an optical system for obtaining optical measurements from the test cartridge; and
a microprocessor that is coupled to the optical system, the microprocessor being adapted to control the electronic reader, receive the optical measurements and determine assay results from the optical measurements.

20. The electronic reader of claim 19, wherein the cartridge further comprises a tab in the slot that provides a force to move the test strip holder of the test cartridge to begin the assay when the test cartridge is inserted into the slot thereby allowing for single user action for performing the assay and measuring the assay results.

21. The electronic reader of claim 20, wherein the slot is vertical to provide a vertical orientation for the test cartridge which aids in movement of the sample along the test strip and the buffer liquid along the buffer membrane and the test strip.

22. The electronic reader of claim 19, wherein the electronic device further comprises a display to show the assay results and/or error information when an error is encountered during the assay or operation of the electronic reader.

23. The electronic reader of claim 19, wherein the electronic device further comprises an NFC reader for reading information about the test cartridge including at least one of a type of assay that is performed by the test cartridge and parameters that are used to determine the assay results from assay measurements.

24. The electronic reader of claim 19, wherein the electronic device further comprises a communication module for pairing and/or communicating with an application of an external device for sending the assay results and associated assay test information.

25. The electronic reader of claim 24, wherein the communication module is adapted to use a Bluetooth or WiFi communication protocol.

26. A kit for performing an assay for point of care testing, wherein the kit comprises:
at least one test cartridge that allows for an automated assay of various analytes in a sample, wherein the test cartridge comprises:
a capillary assembly to receive, store and release the sample;
a buffer capsule for storing and releasing a buffer liquid;
a buffer membrane to receive the buffer liquid during the assay;
a test strip that is coupled to the buffer membrane and adapted to perform the assay after receiving the sample and the buffer liquid;
a test strip holder adapted to hold the test strip and the buffer membrane and move towards the capillary assembly and the buffer membrane to provide the sample to the test strip and the buffer liquid to the buffer membrane when the assay is performed;
an enclosure for providing a housing for the test cartridge; and
an electronic reader for performing an assay test and measuring test results, wherein the electronic reader comprises:
a slot for receiving the at least one test cartridge;
an optical system for obtaining optical measurements from the at least one test cartridge; and
a microprocessor that is coupled to the optical system, the microprocessor being adapted to control the electronic reader, receive the optical measurements and determine assay results from the optical measurements.

27. A method of measuring a sample with at least one analyte of interest, wherein the method comprises:
providing a sample to a test cartridge having a test strip and a buffer membrane for performing an assay on the sample to detect the at least one analyte of interest;
inserting the test cartridge into an electronic reader which provides an actuation force to initiate the assay in the test cartridge;
measuring assay results using the electronic reader; and
displaying, storing and/or sending the assay results using the electronic reader.

28. The method of claim 27, wherein the test cartridge allows for an automated assay of various analytes in a sample, wherein the test cartridge comprises:
a capillary assembly to receive, store and release the sample;
a buffer capsule for storing and releasing a buffer liquid;
a buffer membrane to receive the buffer liquid during the assay;
a test strip that is coupled to the buffer membrane and adapted to perform the assay after receiving the sample and the buffer liquid;
a test strip holder adapted to hold the test strip and the buffer membrane and move towards the capillary assembly and the buffer membrane to provide the sample to the test strip and the buffer liquid to the buffer membrane when the assay is performed; and
an enclosure for providing a housing for the test cartridge.

29. The method of claim 27, wherein the electronic reader is for performing an assay test and measuring test results, wherein the electronic reader comprises:
a slot for receiving a test cartridge that allows for an automated assay of various analytes in a sample, wherein the test cartridge comprises:
a capillary assembly to receive, store and release the sample;
a buffer capsule for storing and releasing a buffer liquid;
a buffer membrane to receive the buffer liquid during the assay;
a test strip that is coupled to the buffer membrane and adapted to perform the assay after receiving the sample and the buffer liquid;
a test strip holder adapted to hold the test strip and the buffer membrane and move towards the capillary assembly and the buffer membrane to provide the sample to the test strip and the buffer liquid to the buffer membrane when the assay is performed;
an enclosure for providing a housing for the test cartridge an optical system for obtaining optical measurements from the test cartridge; and
a microprocessor that is coupled to the optical system, the microprocessor being adapted to control the electronic reader, receive the optical measurements and determine assay results from the optical measurements.
